(19) United States
(12) Patent Application Publication
(10) Pub. No.: US 2023/0402181 A1
(43) Pub. Date: Dec. 14, 2023

(54) METHOD, STORAGE MEDIUM, AND SYSTEM OF SKIN DETECTION FOR OBJECT

(71) Applicant: Alibaba (China) Co., Ltd., Hangzhou (CN)

(72) Inventors: Yuan GAO, Hangzhou (CN); Jing XU, Hangzhou (CN); Yu WANG, Hangzhou (CN); Wei LIU, Hangzhou (CN)

(21) Appl. No.: 18/048,595

(22) Filed: Oct. 21, 2022

(30) Foreign Application Priority Data
Jun. 8, 2022 (CN) 202210641081.8

(51) Int. Cl. G16H 50/20; G06T 7/00

(52) U.S. Cl. CPC G16H 50/20; G06T 7/0012; G06T 2207/30088

(57) ABSTRACT

A method of skin detection for an object includes: acquiring an image of skin covering an outer surface of an object to be detected; evaluating the image of the skin to determine a skin lesion region of the object to be detected, in which the skin lesion region is an image region with a skin lesion feature in the image of the skin; determining a skin lesion attribute of the object to be detected based on the skin lesion feature of the skin lesion region and an object type of the object to be detected, in which the skin lesion attribute is used for describing a skin lesion generated on the object to be detected; and matching lesion data recorded in a lesion database based on the skin lesion attribute to determine a pathological result of the object to be detected.

### CROSS-REFERENCE TO RELATED APPLICATIONS

The present application claims priority to and the benefits of Chinese Patent Application Serial No. 202210641081.8, filed on Jun. 8, 2022, entitled “METHOD, STORAGE MEDIUM, AND PROCESSOR OF SKIN DETECTION FOR OBJECT,” which is incorporated herein by reference in its entirety.

### TECHNICAL FIELD

The present disclosure relates to the field of computers, and in particular, to a method, a storage medium, and a system of skin detection for an object.

### BACKGROUND

At present, for artificial intelligence-assisted dermatosis diagnosis, classification and prediction of dermatoses is usually implemented based on deep learning, so as to determine a disease diagnosis result. However, this method is too rough to accurately interpret the disease, and therefore, there is a problem of a low detection accuracy for an image of skin of an object.

For the above problem, no effective solution has been proposed yet.

### SUMMARY

Embodiments of the present disclosure provide a method of skin detection for an object. In some embodiments, the method includes: acquiring an image of skin covering an outer surface of an object to be detected; evaluating the image of the skin to determine a skin lesion region of the object to be detected, wherein the skin lesion region is an image region with a skin lesion feature in the image of the skin; determining a skin lesion attribute of the object to be detected based on the skin lesion feature of the skin lesion region and an object type of the object to be detected, wherein the skin lesion attribute is used for describing a skin lesion generated on the object to be detected; and matching lesion data recorded in a lesion database based on the skin lesion attribute to determine a pathological result of the object to be detected.

In some embodiments, the method includes: displaying, in response to an image input instruction acting on an operation interface, an image of skin covering an outer surface of an object to be detected from a medical diagnosis platform on the operation interface; and displaying, in response to a detection operation instruction acting on the operation interface, a pathological result of the object to be detected on the operation interface, wherein the pathological result is obtained by matching lesion data recorded in a lesion database based on a skin lesion attribute of the object to be detected, the skin lesion attribute is determined based on a skin lesion feature of a skin lesion region and an object type of the object to be detected, and the skin lesion region is obtained by recognizing the image of the skin.

In some embodiments, the method includes: displaying an image of skin covering an outer surface of an object to be detected on a presentation screen of a virtual reality (VR) device or an augmented reality (AR) device; evaluating the image of the skin to determine a skin lesion region of the object to be detected, wherein the skin lesion region is an image region with a skin lesion feature in the image of the skin sensed by the VR device or the AR device; determining a skin lesion attribute of the object to be detected based on the skin lesion feature of the skin lesion region and an object type of the object to be detected, wherein the skin lesion attribute is used for describing a skin lesion generated on the object to be detected; matching lesion data recorded in a lesion database based on the skin lesion attribute to determine a pathological result of the object to be detected; and driving the VR device or the AR device to display the skin lesion attribute and the pathological result.

In some embodiments, the method includes: acquiring, by calling a first interface, an image of skin covering an outer surface of an object to be detected, wherein the first interface comprises a first parameter, and a parameter value of the first parameter is the image of the skin; evaluating the image of the skin to determine a skin lesion region of the object to be detected, wherein the skin lesion region is an image region with a skin lesion feature in the image of the skin; determining a skin lesion attribute of the object to be detected based on the skin lesion feature of the skin lesion region and an object type of the object to be detected, wherein the skin lesion attribute is used for describing a skin lesion generated on the object to be detected; matching lesion data recorded in a lesion database based on the skin lesion attribute to determine a pathological result of the object to be detected; and outputting, by calling a second interface, the skin lesion attribute and the pathological result, wherein the second interface comprises a second parameter, and a value of the second parameter is the skin lesion attribute and the pathological result.

Embodiments of the present disclosure provide a non-transitory computer-readable medium storing a set of instructions that is executable by at least one processor of an apparatus to cause the apparatus to perform any of the method of skin detection for an object above.

Embodiments of the present disclosure provide a system. The system includes: a memory storing a set of instructions, and one or more processors configured to execute the set of instructions to cause the system to perform any of the method of skin detection for an object above.

It should be understood that the above general descriptions and the following detailed descriptions are merely for exemplary and explanatory purposes, and do not limit the present disclosure.

### BRIEF DESCRIPTION OF THE DRAWINGS

The accompanying drawings described here are intended to provide a further understanding of the present disclosure, and constitute a part of the present disclosure. Illustrative embodiments of the present disclosure and descriptions thereof are intended to explain the present disclosure, and do not constitute an improper limitation to the present disclosure. In the accompanying drawings:

FIG. 1 is a block diagram of an example hardware structure of a computer terminal (or a mobile device) for implementing a method of skin detection for an object according to some embodiments of the present disclosure;

FIG. 2 is a block diagram of an example hardware structure of a virtual reality device for implementing a method of skin detection for an object according to some embodiments of the present disclosure;

FIG. 3 is a flowchart of an example method of skin detection for an object according to some embodiments of the present disclosure;

FIG. 4 is a flowchart of another example method of skin detection for an object according to some embodiments of the present disclosure;

FIG. 5 is a flowchart of another example method of skin detection for an object according to some embodiments of the present disclosure;

FIG. 6 is a flowchart of another example method of skin detection for an object according to some embodiments of the present disclosure;

FIG. 7 is a flowchart of an example clinical dermoscopic diagnosis method based on a transformer model according to some embodiments of the present disclosure;

FIG. 8 is a schematic diagram of an example automated diagnosis system for general dermatosis according to some embodiments of the present disclosure;

FIG. 9 is a schematic diagram of an example apparatus for skin detection for an object according to some embodiments of the present disclosure;

FIG. 10 is a schematic diagram of another example apparatus for skin detection for an object according to some embodiments of the present disclosure;

FIG. 11 is a schematic diagram of another example apparatus for skin detection for an object according to some embodiments of the present disclosure;

FIG. 12 is a schematic diagram of another example apparatus for skin detection for an object according to some embodiments of the present disclosure; and

FIG. 13 is a structural block diagram of an example computer terminal according to some embodiments of the present disclosure.

### DETAILED DESCRIPTION

In order to enable those skilled in the art to better understand the solutions in the present disclosure, the technical solutions in the embodiments of the present disclosure will be described clearly and completely with reference to the accompanying drawings in the embodiments of the present disclosure. It is apparent that the described embodiments are merely some of rather than all the embodiments of the present disclosure. Based on the embodiments of the present disclosure, all other embodiments derived by those of ordinary skill in the art without any creative efforts fall within the protection scope of the present disclosure.

It should be noted that the terms “first,” “second,” and the like in the description and claims of the present disclosure and the above drawings are used for distinguishing similar objects, and are not necessarily used for describing a specific sequence or precedence order. It should be understood that the data so used may be interchanged under appropriate circumstances such that the embodiments of the disclosure described herein can be implemented in sequences other than those illustrated or described herein. Furthermore, the terms “include,” “have,” and any variations thereof are intended to cover non-exclusive inclusion. For example, a process, method, system, product, or device including a series of steps or units is not necessarily limited to those steps or units expressly listed, but can include other steps or units not expressly listed or inherent to the processes, methods, products, or devices.

An image of skin diagnosis system (DermatFormer) may be configured to perform a method of skin detection for an object according to some embodiments of the present disclosure. A Computer Assisted Diagnosis (CAD) module may be configured to assist a doctor in efficient diagnosis and accurate interpretation of a disease. Embodiments of the present disclosure provide methods of skin detection for an object, and storage medium and processors for performing the above methods, to at least solve the technical problem of a low detection accuracy and precision for an image of skin of an object.

In the embodiments of the present disclosure, an image of skin covering an outer surface of an object to be detected is acquired; the image of the skin is evaluated to determine a skin lesion region of the object to be detected, in which the skin lesion region is an image region with a skin lesion feature in the image of the skin; a skin lesion attribute of the object to be detected is determined based on the skin lesion feature of the skin lesion region and an object type of the object to be detected, in which the skin lesion attribute is used for describing a skin lesion generated on the object to be detected; and a pathological result of the object to be detected is determined, based on the skin lesion attribute, by matching lesion data recorded in a lesion database. In other words, in the embodiments of the present disclosure, by processing the acquired skin image, extracting the skin lesion region, matching the skin lesion attribute of the determined skin lesion region with the lesion data recorded in the lesion database, and determining an attribute feature of a disease, an objective of accurately determining the pathological result can be achieved, thereby realizing the technical effect of improving the detection accuracy and precision of the image of the skin of the object. Thus, the image detection methods provided by the embodiments of the present disclosure solve the technical problem of the low detection accuracy and precision for an image of skin of an object.

According to some embodiments of the present disclosure, a method of skin detection for an object is further provided. It should be noted that the steps shown in the flowchart of the accompanying drawing may be performed in a computer system such as a set of computer-executable instructions. Moreover, although a logical order is shown in the flowchart, in some cases, steps shown or described may be performed in an order different from that herein.

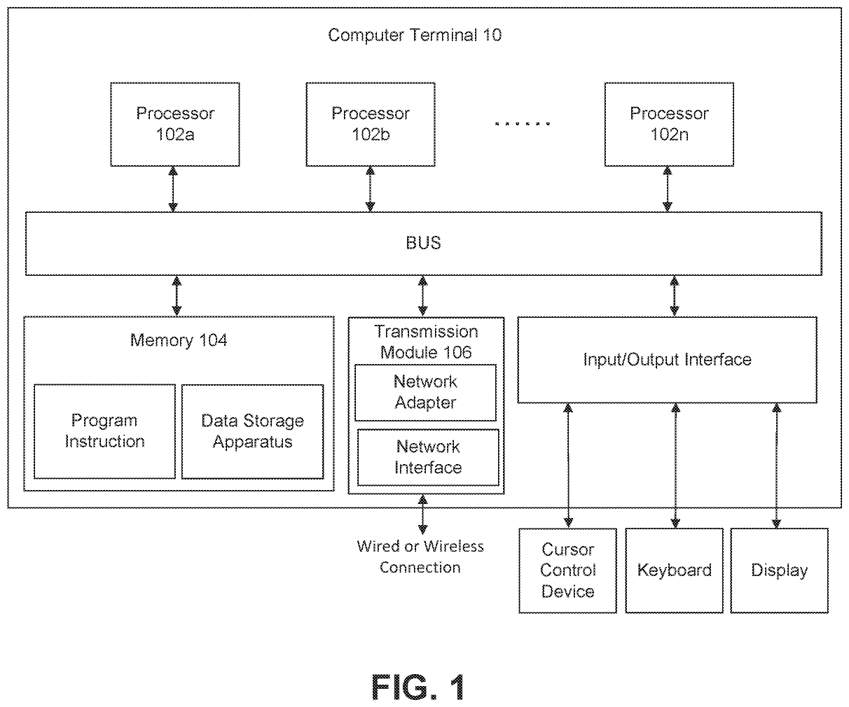

FIG. 1 is a block diagram of a hardware structure of a computer terminal (or a mobile device) for implementing a method of skin detection for an object according to some embodiments of the present disclosure. As shown in FIG. 1, computer terminal 10 (or mobile device 10) may include one or more processors (e.g., processors 102a, 102b, . . . , 102n in the drawing), memory 104 configured to store data, and a transmission module 106 configured to provide communication functions. The processor(s) may include, but is not limited to, a processing apparatus such as a microprocessor (MCU) or a programmable logic device, e.g., a Field Programmable Gate Array (FPGA), a hardware accelerator, etc. In addition, the computer terminal or mobile device 10 may also include a display, an input/output interface (I/O interface), a universal serial bus (USB) port (which may be included as one of ports of a BUS), a network interface, a power supply, or a camera. Those of ordinary skill in the art may understand that the structure shown in FIG. 1 is merely illustrative, which does not limit the structure of the above electronic apparatus. For example, computer terminal 10 may also include more or fewer components than that shown in FIG. 1, or have a different configuration from that shown in FIG. 1.

It should be noted that the one or more processors described above or other skin detection circuits for an object may generally be referred to herein as a “skin detection circuit for an object.” The skin detection circuit for an object may be embodied in whole or in part as software, hardware, firmware, or any other combination. Additionally, the skin detection circuit for an object may be a single stand-alone processing module, or incorporated in whole or in part into any of other elements in the computer terminal or mobile device 10. As involved in some embodiments of the present disclosure, the skin detection circuit for an object acts as a processor to control, for example, the selection of a variable resistance terminal path connected to an interface.

Memory 104 may be configured to store software programs and modules of application software, and act as a program instructions/data storage apparatus corresponding to the method of skin detection for an object in some embodiments of the present disclosure. The processor performs various functional applications and the method of skin detection for an object by running the software programs and modules stored in memory 104. Memory 104 may include a high-speed random access memory, and may also include a non-volatile memory, such as one or more magnetic storage apparatuses, a flash memory, or another non-volatile solid-state memory. In some examples, memory 104 may further include memories remotely arranged with respect to the processor, and these remote memories may be connected to computer terminal 10 through a network. Examples of the above network include, but are not limited to, the Internet, an intranet, a local area network, a mobile communication network, and any combination thereof.

Transmission apparatus 106 is configured to receive or send data via a network. Specific examples of the above network may include a wireless network provided by a communication provider of computer terminal 10. In some examples, transmission apparatus 106 includes a Network Interface Controller (NIC), which may be connected to other network devices through a base station so as to communicate with the Internet. In some examples, transmission apparatus 106 may be a Radio Frequency (RF) module, which is configured to communicate with the Internet wirelessly.

The display may be, for example, a touch screen-type liquid crystal display (LCD), and the liquid crystal display enables a user to interact with a user interface of computer terminal or mobile device 10.

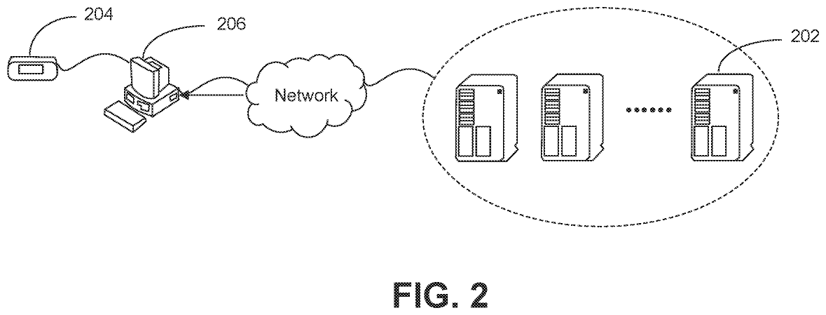

FIG. 2 is a block diagram of a hardware structure of a virtual reality device for implementing a method of skin detection for an object according to some embodiments of the present disclosure. As shown in FIG. 2, virtual reality device 204 is connected to terminal 206, and terminal 206 and server 202 are connected through a network. The above virtual reality device 204 is not limited to a virtual reality helmet, virtual reality glasses, a virtual reality all-in-one machine, and the like. The above terminal 206 is not limited to a PC, a mobile phone, a tablet computer, and the like. Server 202 may be a server corresponding to a media content operator, and the above network includes, but is not limited to, a wide area network, a metropolitan area network, or a local area network.

Optionally, virtual reality device 204 in some embodiments includes a memory, a processor, and a transmission apparatus. The memory is configured to store an application program. The application program may be configured to perform acquiring an image of skin covering an outer surface of an object to be detected; evaluating the image of the skin to determine a skin lesion region of the object to be detected, the skin lesion region being an image region with a skin lesion feature in the image of the skin; determining a skin lesion attribute of the object to be detected based on the skin lesion feature of the skin lesion region and an object type of the object to be detected, the skin lesion attribute being used for describing a skin lesion generated on the object to be detected; and matching lesion data recorded in a lesion database based on the skin lesion attribute to determine a pathological result of the object to be detected, thereby solving the technical problem of a low detection accuracy for an image of skin of an object, and achieving the technical effect of improving the accuracy of performing skin image detection on an object.

The terminal in some embodiments may be configured to display an image of skin covering an outer surface of an object to be detected on a presentation screen of a Virtual Reality (VR) device or an Augmented Reality (AR) device; recognize the image of the skin, and determine a skin lesion region of the object to be detected, the skin lesion region being an image region with a skin lesion feature in the image of the skin sensed by virtual reality device 204; determine a skin lesion attribute of the object to be detected based on the skin lesion feature of the skin lesion region and an object type of the object to be detected, the skin lesion attribute being used for describing a skin lesion generated on the object to be detected; match lesion data recorded in a lesion database based on the skin lesion attribute to determine a pathological result of the object to be detected; and drive the VR device or AR device/virtual reality device 204 to display the skin lesion attribute and the pathological result.

Optionally, an eye-tracking Head Mount Display (HMD) and an eye-tracking module provided in virtual reality device 204 in some embodiments provide the same functions as those in the foregoing embodiments. That is, a screen in the HMD is configured to display a real-time image, and the eye-tracking module in the HMD is configured to acquire a real-time eyeball movement path of a user. The terminal in some embodiments acquires position information and motion information of a user in the three-dimensional reality space through a tracking system, and calculates three-dimensional coordinates of the head of the user in a virtual three-dimensional space and a field of view orientation of the user in the virtual three-dimensional space.

The block diagram of the hardware structure shown in FIG. 2 may be used not only as an example block diagram of the above AR/VR device (or mobile device), but also as an example block diagram of the above server.

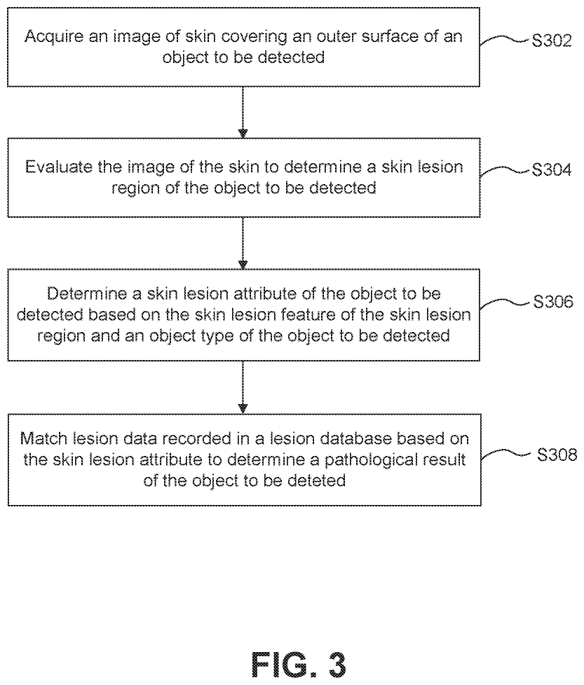

In the above operating environment, a method of skin detection for an object as shown in FIG. 3 is provided in the present disclosure. It should be noted that, the method of skin detection for an object in some embodiments may be performed by the mobile terminal of the embodiments shown in FIG. 1.

FIG. 3 is a flowchart of a method of skin detection for an object according to some embodiments of the present disclosure. As shown in FIG. 3, the method may include the following steps S302, S304, S306, and S308.

In step S302, an image of skin covering an outer surface of an object to be detected is acquired.

In the technical solution provided in the above step S302 of some embodiments of the present disclosure, the image of the skin covering the outer surface of the object to be detected is acquired. The object to be detected may be patients of different ages. For example, the patients of different ages may be infants, or may be elderly persons, which are only used for illustration here, and the present disclosure is not limited thereto.

For example, the image of the skin covering the outer surface of the object to be detected may be an image of skin uploaded by a patient from a client terminal, or an image of skin obtained by a dermatologist in clinical practice.

In step S304, the image of the skin is evaluated to determine a skin lesion region of the object to be detected. The skin lesion region is an image region with a skin lesion feature in the image of the skin.

In the technical solution provided in the above step S304 of some embodiments of the present disclosure, the acquired skin image is recognized, and the skin lesion region of the object to be detected is determined. The skin lesion region may be an image region with a skin lesion feature in the image of the skin. For example, it may be a skin lesion disease region, a discriminating region of the skin disease, a disease region, a skin lesion target region, a site with a nidus, and the like. The above are merely examples, and not meant to limit the present disclosure. The skin lesion feature may be an image feature obtained after redundant features are removed from a skin picture.

In step S306: a skin lesion attribute of the object to be detected is determined based on the skin lesion feature of the skin lesion region and an object type of the object to be detected. The skin lesion attribute is used for describing a skin lesion generated on the object to be detected.

In the technical solution provided in the above step S306 of some embodiments of the present disclosure, according to the obtained skin lesion region of the object to be detected, the skin lesion feature of the skin lesion region is obtained, and the skin lesion attribute of the skin lesion region is determined in combination with the type of the object to be detected. The skin lesion attribute may be an attribute feature of the diseased skin lesion, which may be used as evidence for interpretability of a disease result of the object to be detected. For example, the skin lesion attribute may include redness and swelling, scurf, hyperpigmentation, and the like, which are merely examples and not meant to limit the present disclosure. The object type may be the skin of different body parts. For example, the skin of different body parts may be leg skin and facial skin, which are merely examples and not meant to limit the present disclosure.

In step S308, lesion data recorded in a lesion database is matched based on the skin lesion attribute to determine a pathological result of the object to be detected.

In the technical solution provided by the above step S308 of some embodiments of the present disclosure, the determined skin lesion attribute of the object to be detected is matched with the acquired lesion data in the lesion database, so as to determine the pathological result of the object to be detected. The pathological result of the object to be detected may be a result of the disease diagnosis for the skin lesion region of the object to be detected. For example, the pathological result may be a type of disease, a classification of dermatosis, and the like, which are merely examples and not meant to limit the present disclosure. The lesion database may be a statistical library used for storing lesion types and skin lesion attribute sets, such as a lesion data set library calibrated using prior knowledge of dermatologists, which is merely an example and not meant to limit the present disclosure.

Through the above step S302 to step S308 of some embodiments of the present disclosure, an image of skin covering an outer surface of an object to be detected is acquired; the image of the skin is evaluated to determine a skin lesion region of the object to be detected, the skin lesion region being an image region with a skin lesion feature in the image of the skin; a skin lesion attribute of the object to be detected is determined based on the skin lesion feature of the skin lesion region and an object type of the object to be detected, the skin lesion attribute being used for describing a skin lesion generated on the object to be detected; and a pathological result of the object to be detected is determined, based on the skin lesion attribute, by matching lesion data recorded in a lesion database. In other words, in some embodiments of the present disclosure, by processing the acquired skin image, extracting the skin lesion disease region, matching the skin lesion attribute of the determined skin lesion region with the lesion data recorded in the lesion database, and determining an attribute feature of a disease, an objective of accurately determining the pathological result is achieved, thereby achieving the technical effect of improving the detection accuracy of the image of the skin of the object. Further, the image detection methods provided by the embodiments of the present disclosure solve the technical problem of a low detection accuracy for the image of skin of the object.

The above method of the embodiments will be further described below.

In some embodiments, step S304 of evaluating the image of the skin to determine the skin lesion region of the object to be detected includes: recognizing the image of the skin to obtain a region token sequence, each region token in the region token sequence being used for representing a skin region of the object to be detected; and extracting a sub-region token sequence from the region token sequence, each region token in the sub-region token sequence being used for representing a sub-skin lesion region in the skin lesion region. A region token is a region token of an image after processing.

In some embodiments, the acquired image of the skin is recognized to obtain the region token sequence of the image of the skin, the sub-region token sequence is extracted from the obtained region token sequence, and the sub-skin lesion region is determined based on each region token in the extracted sub-region token sequence, thereby determining the skin lesion region of the object to be detected. The region token sequence may be a local token sequence obtained based on the acquired image of the skin, and each region token in the region token sequence may be used for representing a skin region of the object to be detected. For example, the region token in the region token sequence may be a local token obtained based on the acquired skin image. The skin region may be represented by a position vector and a position code of the region token. The sub-region token sequence may be a partial token sequence in the region token sequence. For example, the sub-region token sequence may be a token result obtained by removing redundant features from a token sequence of the skin lesion region. Each region token in the sub-region token sequence is used for representing a sub-skin lesion region in the skin lesion region. For example, the region tokens in the sub-region token sequence may be localized tokens associated with niduses.

In some embodiments, extracting the sub-region token sequence from the region token sequence includes: converting the region token sequence into an image feature sequence of the image of the skin, an image feature in the image feature sequence being used for representing the skin region corresponding to the region token in the region token sequence; determining a sub-image feature sequence corresponding to the skin lesion regions from the image feature sequence, the skin lesion region including the skin region corresponding to the image feature in the sub-image feature sequence; and determining the sub-region token sequence corresponding to the sub-image feature sequence.

In some embodiments, based on the obtained region token sequence of the image of the skin, the image feature sequence of the corresponding skin image is generated, and the sub-image feature sequence corresponding to the skin lesion region is determined from the generated image feature sequence, so as to determine the sub-region token sequence corresponding to the sub-image feature sequence. The image feature in the image feature sequence may be used for representing the skin region corresponding to the region token in the region token sequence, and the skin lesion region may include the skin region corresponding to the image feature in the sub-image feature sequence.

Optionally, the region token sequence of the image of the skin may be an image feature sequence of a corresponding skin image generated by a Multi-head Self-Attention (MSA) module. A sub-image feature sequence corresponding to the skin lesion region may be extracted from the generated image feature sequence. A Multi-head Self-Attention (MSA) module may be configured to generate an image feature sequence based on a token sequence.

In some embodiments, extracting the sub-region token sequence from the region token sequence includes: determining at least one region token having an importance level higher than a target threshold in the region token sequence as the sub-region token sequence. The importance level may be used for representing a level of importance of the corresponding region token to the pathological result.

In some embodiments, the importance level of each region token in the region token sequence is calculated. The importance level of each region token is compared with a preset target threshold. When there is at least one region token having the importance level higher than the target threshold, the region token can be determined as the sub-region token sequence.

Optionally, the at least one region token may also be first K tokens selected after sorting the region tokens in the region token sequence according to their respective importance levels.

In some embodiments, determining at least one region token having the importance level higher than the target threshold in the region token sequence as the sub-region token sequence includes: selecting the at least one region token having the importance level higher than the target threshold from the region token sequence based on a Lesion Token Selection (LTS) module. The LTS module is at least configured to determine the importance level. A Lesion Token Selection (LTS) module may be configured to automatically extract a token sequence of a skin lesion region to remove redundant features or guide an encoder of a Vision Transformer (ViT) to select localized tokens related to the lesion at different levels. A Vision Transformer (ViT) model may be configured to partition an input image into multiple local image blocks, apply linearly embedding to each local image block, and send a resulting vector sequence to a standard encoder for learning.

In some embodiments, a region token sequence is fed to the lesion token selection module to select at least one region token having the importance level higher than the target threshold from the region token sequence. The lesion token selection module may at least be configured to determine an importance level of each region token in the region token sequence, and the target threshold may be a preset target importance level threshold.

Optionally, the lesion token selection module may be configured to learn more unique features using spatial information of the lesion and provide a visual evidence of the nidus location.

In some embodiments, generating the region token sequence for the image of the skin includes: partitioning the image of the skin into multiple region tokens based on a vision transformer model to obtain the region token sequence, the vision transformer model being obtained by training based on a self-attention mechanism.

In some embodiments, the image of the skin may be divided into multiple region tokens by the vision transformer model, and the obtained multiple region tokens may form the region token sequence. The vision transformer model may be obtained by training based on the self-attention mechanism, and may be configured for end-to-end training and inference. The training based on the self-attention mechanism may be training based on the multi-head self-attention module.

Optionally, partitioning the image of the skin into the multiple region tokens may be based on a mesh processing to the image of the skin to generate the multiple region tokens.

In some embodiments, determining the skin lesion attribute of the object to be detected based on the skin lesion feature of the skin lesion region and the object type of the object to be detected includes: fusing the skin lesion features of the multiple sub-skin lesion regions corresponding to the sub-region token sequence with the object type to obtain the skin lesion attribute.

In some embodiments, the skin lesion features of the multiple sub-skin lesion regions corresponding to the region token sequence and the object type are fused. The skin lesion attribute is determined based on a fusion result. The skin lesion features of the multiple sub-skin lesion regions and the object type may be fused through a contextual fusion module and a local fusion module. The contextual fusion module and the local fusion module may further be configured to learn an interaction relationship between the region token sequence and the selected sub-region token sequence. A Contextual Fusion Module (CFM) may be configured to fuse global context information. A Local Fusion Module (LFM) may be configured to fuse fine-grained local features.

In some embodiments, the method further includes: calibrating the skin lesion attribute based on a target skin lesion attribute of the object to be detected, or calibrating the pathological result based on a target pathological result of the object to be detected, in order to match the skin lesion attribute with the pathological result.

In some embodiments, the skin lesion attribute of the determined object to be detected is calibrated by determining the target skin lesion attribute of the object to be detected, or the pathological result of the determined object to be detected is calibrated by determining the target pathological result of the object to be detected, thus achieving an objective of matching the skin lesion attribute and the pathological result.

Optionally, the target skin lesion attribute of the object to be detected may be a skin lesion attribute pre-calculated based on prior knowledge, and the target pathological result of the object to be detected may be a disease type pre-calculated based on the prior knowledge. Calibrations on the skin lesion attribute and the pathological result may be achieved by a bilateral prediction distillation module. A Bilateral Prediction Distillation (BPD) module may be configured to correct misclassified samples by using a pre-computed co-occurrence matrix of disease and skin lesion attributes.

In the embodiments of the present disclosure, the image of the skin is partitioned into multiple region tokens based on the vision transformer model to obtain the region token sequence, the region token sequence is converted into the image feature sequence of the image of the skin, and the sub-image feature sequence corresponding to the skin lesion region is determined from the image feature sequence, so as to obtain the sub-region token sequence corresponding to the sub-image feature sequence. The skin lesion features of multiple sub-skin lesion regions corresponding to the sub-region token sequence and the object type are fused to obtain the skin lesion attribute. Calibration is performed to match the skin lesion attribute and the pathological result. In other words, the embodiments of the present disclosure obtains the region token sequence based on the vision transformer model, extracts the sub-region token sequence therefrom, and fuses data information corresponding to the sub-region token sequence, so as to achieve an accurate matching between the skin lesion attribute and the pathological result. Therefore, the technical effect of improving the detection accuracy of the image of the skin of the object is achieved, Furthermore, the image detection method provided by the embodiments of the present disclosure solves the technical problem of a low detection accuracy for an image of skin of an object.

The embodiments of the present disclosure further provide another method of skin detection for an object from the side of human-machine interaction.

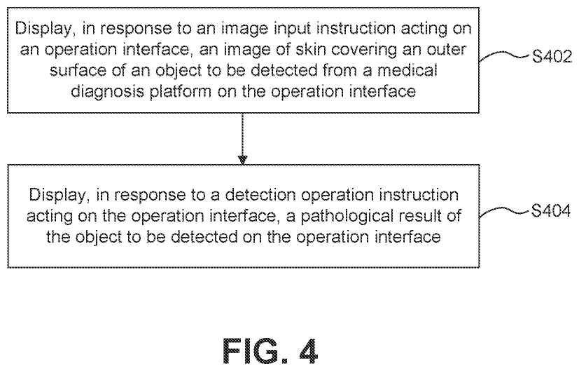

FIG. 4 is a flowchart of another method of skin detection for an object according to some embodiments of the present disclosure. As shown in FIG. 4, the method may include the following steps S402 and S404.

In step S402, in response to an image input instruction acting on an operation interface, an image of skin covering an outer surface of an object to be detected from a medical diagnosis platform is displayed on the operation interface.

In the technical solution provided in the above step S402 in some embodiments of the present disclosure, the image input instruction acts on the operation interface. The operation interface responds to the instruction to display, on the operation interface, the image of the skin covering the outer surface of the object to be detected from the medical diagnosis platform.

In some embodiments, the image input instruction may be used for inputting image data of the skin on the outer surface of the object to be detected, for example, by issuing, on the operation interface, an instruction of inputting the image of a patient's face skin. The input of the image of a patient's face skin can be realized in response to the instruction.

In step S404, in response to a detection operation instruction acting on the operation interface, a pathological result of the object to be detected is displayed on the operation interface. The pathological result is obtained by matching lesion data recorded in a lesion database based on a skin lesion attribute of the object to be detected. The skin lesion attribute is determined based on a skin lesion feature of a skin lesion region and an object type of the object to be detected. The skin lesion region is obtained by recognizing the image of the skin.

In the technical solution provided by the above step S404 in some embodiments of the present disclosure, the detection operation instruction acts on the operation interface. The operation interface responds to the instruction, and the pathological result of the object to be detected is displayed on the operation interface. The pathological result may be a disease diagnosis of the skin lesion region of the object to be detected obtained, based on the skin lesion attribute of the object to be detected, by matching the lesion data recorded in the lesion database. The skin lesion attribute may be determined based on the skin lesion feature of the skin lesion region and the object type of the object to be detected. The skin lesion region may be an image region with the skin lesion feature obtained by recognizing the image of the skin of the object to be detected.

In some embodiments, the detection operation instruction may be used for outputting the pathological result data of the object to be detected. For example, by issuing, on the operation interface, an instruction of outputting the pathological result data of the object to be detected, the output of the pathological result of the object to be detected is realized in response to the instruction.

Optionally, the pathological result of the object to be detected may be used for generating a case report, and providing physicians with more reliable and interpretable diagnostic results.

In the embodiments of the present disclosure, based on the image input instruction and the detection operation instruction acting on the operation interface, the image of the skin from the medical diagnosis platform and the pathological result of the object to be detected are displayed on the operation interface, which provides an evidence for interpretability of the pathological result, achieves the objective of improving the confidence of the diagnosis result, thereby achieving the technical effect of improving the detection accuracy of the image of the skin of the object. Furthermore, the image detection method provided by the embodiments of the present disclosure solves the technical problem of a low detection accuracy for an image of skin of an object.

The embodiments of the present disclosure further provide another method of skin detection for an object from an application scenario side.

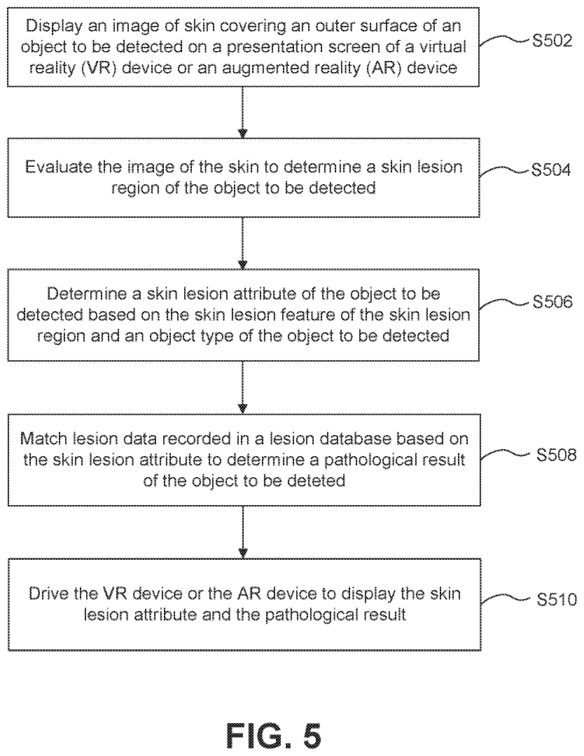

FIG. 5 is a flowchart of another method of skin detection for an object according to some embodiments of the present disclosure. As shown in FIG. 5, the method may include the following steps S502, S504, S506, S508, and S510.

In step S502, an image of skin covering an outer surface of an object to be detected is displayed on a presentation screen of a virtual reality (VR) device or an augmented reality (AR) device.

In the technical solution provided in the above step S502 in some embodiments of the present disclosure, the image of the skin covering the outer surface of the object to be detected is acquired, and the image of the skin is displayed on the presentation screen of the virtual reality (VR) device or the augmented reality (AR) device.

In step S504, the image of the skin is evaluated to determine a skin lesion region of the object to be detected. The skin lesion region is an image region with a skin lesion feature in the image of the skin sensed by the VR device or AR device.

In the technical solution provided by the above step S504 in some embodiments of the present disclosure, determining the skin lesion region in the acquired skin image based on the VR device or the AR device may be determining the image region with the skin lesion feature in the image of the skin.

In step S506, a skin lesion attribute of the object to be detected is determined based on the skin lesion feature of the skin lesion region and an object type of the object to be detected. Determining the skin lesion attribute of the object to be detected may be determining a skin lesion generated on the object to be detected.

In the technical solution provided in the above step S506 in some embodiments of the present disclosure, the skin lesion feature of the skin lesion region is acquired based on the determined skin lesion region. The object type of the object to be detected is acquired based on the object to be detected. Then, the skin lesion attribute of the object to be detected is determined based on the acquired skin lesion feature of the skin lesion region and the object type of the object to be detected. The skin lesion attribute may be used for describing the skin lesion generated on the object to be detected.

Optionally, acquiring the skin lesion feature of the skin lesion region based on the determined skin lesion region may be performing image processing on the determined skin lesion region to obtain an image feature corresponding to the skin lesion region.

Optionally, acquiring the object type of the object to be detected may be obtaining the object type by analyzing the image of the skin of the object to be detected based on deep learning, or may be obtaining the object type based on data information pre-input from a user terminal.

In step S508, lesion data recorded in a lesion database is matched based on the skin lesion attribute to determine a pathological result of the object to be detected.

In the technical solution provided by the above step S508 in some embodiments of the present disclosure, the determined skin lesion attribute is matched with the lesion data recorded in the lesion database, and the pathological result of the object to be detected is determined based on an obtained matching result.

Optionally, the matching of the skin lesion attribute and the lesion data recorded in the lesion database may be based on a calculation of posterior distribution. The obtained matching result may be a loss function obtained by the calculation.

In step S510, the VR device or AR device is driven to display the skin lesion attribute and the pathological result.

In the technical solution provided by the above step S510 in some embodiments of the present disclosure, the VR device or AR device is driven, and the determined skin lesion attribute and pathological result are displayed through the VR device or AR device.

Optionally, driving the VR device or AR device may be achieved by sending a drive signal to the VR device or AR device.

For example, after the skin lesion attribute and the pathological result are determined, a drive signal may be sent from a user terminal, or a drive signal may be sent from a doctor terminal. In response to the drive signal, a display interface of the VR device or AR device displays the determined skin lesion attribute and pathological result.

In the embodiments of the present disclosure, by recognizing the image of the skin displayed on the presentation screen of the VR device or AR device, the skin lesion region of the object to be detected is first determined. Then, the skin lesion attribute of the object to be detected is determined. The pathological result of the object to be detected is obtained through the matching result of the skin lesion attribute and the lesion data. Finally, the VR device or AR device is driven to display the skin lesion attribute and the pathological result, thereby achieving the technical effect of improving the accuracy of detecting the image of the skin of the object. Furthermore, the image detection method provided in the embodiments of the present disclosure solves the technical problem of a low detection accuracy for an image of skin of an object.

The embodiments of the present disclosure further provide another method of skin detection for an object from an interaction side.

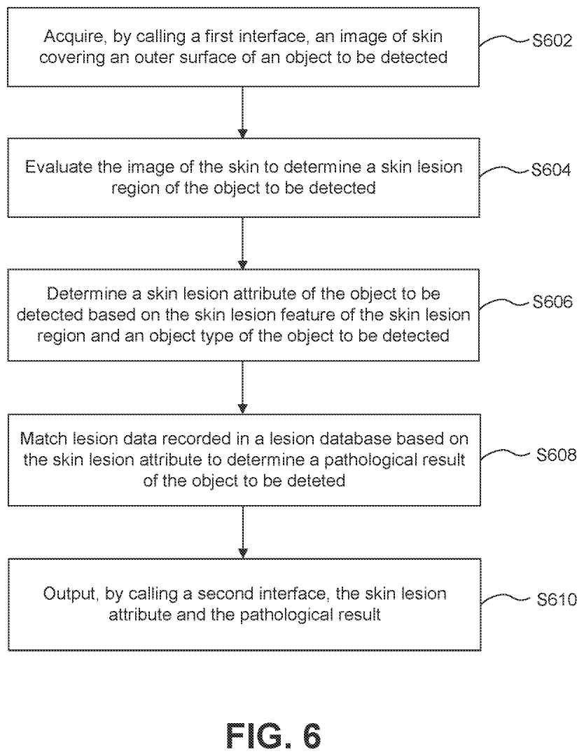

FIG. 6 is a flowchart of another method of skin detection for an object according to some embodiments of the present disclosure. As shown in FIG. 6, the method may include the following steps S602, S604, S606, S608, and S610.

In step S602, by calling a first interface, an image of skin covering an outer surface of an object to be detected is acquired. The first interface includes a first parameter, and a parameter value of the first parameter is the image of the skin.

In the technical solution provided by the above step S602 in some embodiments of the present disclosure, the first interface may be an interface for data interaction between a server and a client. The client may transmit skin image information into the first interface as a first parameter of the first interface, thereby achieving the purpose of acquiring the image of the skin information.

In step S604, the image of the skin is evaluated to determine a skin lesion region of the object to be detected. The skin lesion region is an image region with a skin lesion feature in the image of the skin.

In step S606, a skin lesion attribute of the object to be detected is determined based on the skin lesion feature of the skin lesion region and an object type of the object to be detected. The skin lesion attribute is used for describing a skin lesion generated on the object to be detected.

In step S608, lesion data recorded in a lesion database is matched based on the skin lesion attribute to determine a pathological result of the object to be detected.

In step S610, by calling a second interface, the skin lesion attribute and the pathological result are output. The second interface includes a second parameter, and a value of the second parameter is the skin lesion attribute and the pathological result.

In the technical solution provided by the above step S610 in some embodiments of the present disclosure, the second interface may be an interface for data interaction between the server and the client. The server may call the second interface to enable the terminal device to sequentially output the skin lesion attribute and the pathological result as a parameter of the second interface, for the purpose of providing an evidence for interpretability of the pathological result.

In the embodiments of the present disclosure, the image of the skin covering the outer surface of the object to be detected is acquired by calling the first interface. The image of the skin is evaluated to determine the skin lesion region of the object to be detected. The skin lesion attribute of the object to be detected is determined based on the skin lesion feature of the skin lesion region and the object type of the object to be detected. The pathological result of the object to be detected is determined, based on the skin lesion attribute, by matching the lesion data recorded in the lesion database. The skin lesion attribute and the pathological result are output by calling the second interface. In other words, in the present disclosure, by extracting the skin lesion disease region, and matching the skin lesion attribute of the determined skin lesion region with the lesion data recorded in the lesion database, the attribute feature of the disease can be determined to achieve the objective of accurately determining the pathological result, thereby achieving the technical effect of improving the detection accuracy of the image of the skin of the object. Furthermore, the image detection method provided by the embodiments of the present disclosure solves the problem of a low detection accuracy for an image of skin of an object.

It should be noted that, for the sake of a simple description, the foregoing method embodiments are expressed as a series of action combinations, but those skilled in the art would understand that the present disclosure is not limited to the described action sequence. According to some embodiments of the present disclosure, certain steps may be performed in another order or simultaneously. Secondly, those skilled in the art would also understand that the embodiments described in the specification are exemplary embodiments, and the actions and modules involved are not necessarily required in some embodiments of the present disclosure.

From the description of the above implementations, those skilled in the art can clearly understand that the method of skin detection for an object according to the above embodiments may be implemented by means of software and a hardware platform, and may also be implemented by hardware. Based on the above understanding, the technical solutions of the present disclosure essentially, or the part making contributions to the prior art, can be embodied in the form of a software product. The computer software product is stored in a storage medium (such as a ROM/RAM, a magnetic disk, or an optical disc), and includes several instructions used for causing a terminal device (which may be a mobile terminal, a computer, a server, a network device, or the like) to perform the methods in various embodiments of the present disclosure.

An exemplary implementation of the above method of the embodiments will be further introduced below. Specifically, a method of skin detection for an object will be described.

In a related technology, in order to alleviate the pressure of dermatosis diagnosis, an auxiliary diagnosis system is usually designed based on the artificial intelligence technology to help doctors perform an efficient diagnosis. The auxiliary system mainly functions to learn classification and prediction of dermatoses and directly output a disease diagnosis result.

In the related technology, an auxiliary diagnosis platform for common dermatoses is provided. The platform belongs to a user-terminal oriented (to consumer, “to C”) online consultation platform. A user shoots and uploads an image of the skin condition, and the platform provides a diagnosis result and popular science about the diagnosis result for the image of the skin condition uploaded by the user. However, the platform only outputs a result with the highest probability, which does not conform to the diagnosis logic of a doctor, so there is a problem that the algorithm is not accurate enough.

In another related technology, a method for recognizing skin by artificial intelligence is provided. The method directly provides a result based on an image feature, but only a single diagnostic result is output without any evidence for interpretability. Therefore, there is a problem of low confidence in the diagnosis result.

In another related technology, a method for auxiliary diagnosis of dermatoses is provided. The method is based on a Convolutional Neural Network (CNN), and extracts dependency relationship between local features and long-distance context information, locates discriminative regions of skin lesions using a class activation map, extracts a local target candidate region (region of interest, ROI) from the detected regions, and is fed to a linear classifier head for a second time by combining with global contexts and fused representation, so as to achieve the purpose of predicting the distribution of diseases. However, the algorithm has problems of low computing efficiency and significant amount of occupied computing resources.

In order to solve the above problems, the present embodiments propose a method of skin detection for an object, which has both a user terminal-oriented port and an enterprise-oriented port (to business, “to B”), and provides multiple diagnosis results and a sorting of the confidence of the multiple diagnosis results, which conforms to the doctor's diagnosis process, including the exclusion or confirmation of the disease. In addition, the present embodiments further describe a skin lesion attribute in detail, which increases the confidence of the diagnosis result and further solves the technical problem in improving the accuracy of dermatosis diagnosis.

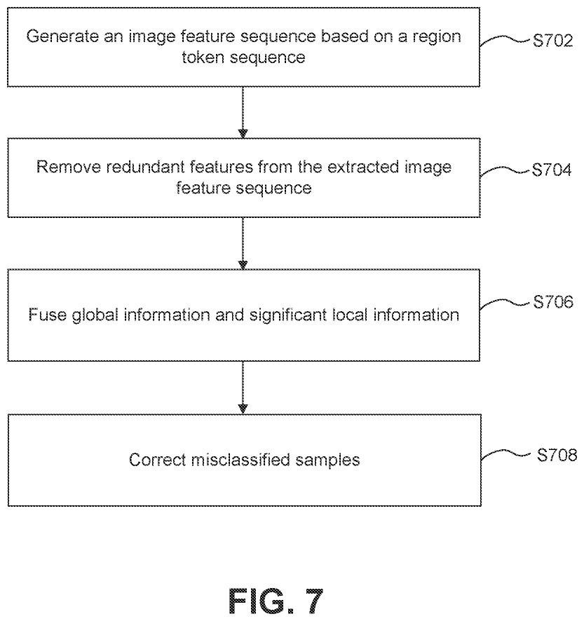

FIG. 7 is a flowchart of a clinical dermoscopic diagnosis method based on a transformer model according to some embodiments of the present disclosure. As shown in FIG. 7, the method may include the following steps S702, S704, S706, and S708.

In step S702, an image feature sequence is generated based on a region token sequence.

In some embodiments, a corresponding image feature sequence is generated based on an extracted region token sequence. The region token sequence may be obtained by a meshing process to an image of skin or an input image, or may be obtained from the input image based on a vision transformer model. Each local token has a specific position code.

Optionally, generating the corresponding image feature sequence based on the extracted region token sequence may be inputting the region token sequence into a multi-head self-attention module, thereby generating the image feature sequence.

In step S704, redundant features are removed from the extracted image feature sequence.

In some embodiments, the extracted image feature sequence is input into a lesion token selection module to remove redundant image features in the image feature sequence. The lesion token selection module may automatically extract the token sequence of the skin lesion region to remove the redundant features.

In step S706, global information and significant local information are fused.

In some embodiments, the global information and the significant local information are fused based on a contextual fusion module and a local fusion module. The global information may be the extracted region token sequence, and the significant local information may be an acquired sub-region token sequence.

Optionally, after fusing the global information and the significant local information, the method may further include classifying conditions and attributes through two independent self-attention modules.

In step S708, misclassified samples are corrected.

In some embodiments, correcting the misclassified samples may be based on a bilateral prediction distillation module. The misclassified samples are corrected by acquiring a pre-computed co-occurrence matrix of disease types and skin lesion attributes.

Some embodiments of the present disclosure are based on the vision transformer model. First, an image feature sequence is generated from a region token sequence, and redundant features are removed from the extracted image feature sequence. Then, global information and significant local information are fused. Finally, misclassified samples are corrected to achieve the purpose of enhancing the learning ability of skin lesion features under different distributions, thereby achieving the technical effect of improving the accuracy of dermatosis diagnosis. Furthermore, the image detection method provided by the embodiments of the present disclosure solves the technical problem of a low accuracy of dermatological diagnosis due to the complexity of skin lesion attributes.

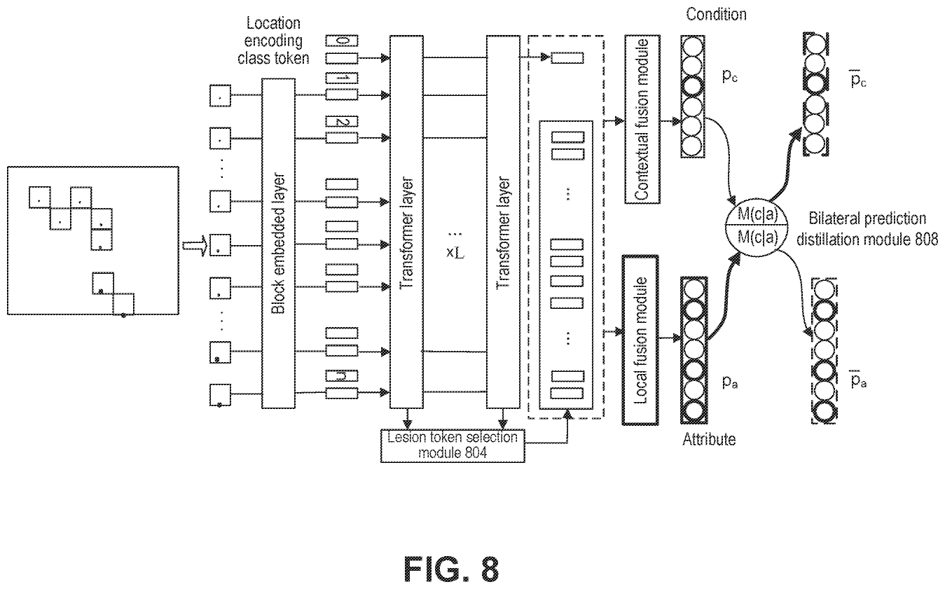

FIG. 8 is a schematic diagram of an automated diagnosis system for general dermatosis according to some embodiments of the present disclosure. It should be noted that the automated diagnosis system for general dermatosis may be configured to perform the above clinical dermoscopic diagnosis method based on a transformer model. As shown in FIG. 8, the system may include the following modules of a network backbone module, a lesion token selection module 804, a disease attribute joint learning module, and a bilateral prediction distillation module 808. It is appreciated that these units/modules may be hardware components or software components stored in memory (e.g., memory 104) for processing by one or more processors (e.g., processors 102a, 102b, 102n).

Network backbone module is configured to partition an input image into multiple region tokens, denoted as {Xₙ, n∈{1, 2, . . . , Nₚ}}, according to a vision transformer model, where:

Nₚ=H×W/P² (1)

In the above formula (1), Nₚ represents the quantity of region tokens, and p represents a side length of the region token.

Local image blocks are flattened and linearly mapped to obtain a feature token of the region token, denoted as x′ₙ. Each local token is also given a learnable position vector to retain encoding information of the location, and at the same time, a class token, denoted as t*₀, is introduced, which may be used for recording global feature information.

The region token sequence of the input image is denoted as {tₙ∈R^{D},n∈{0, 1, . . . , Nₚ}}. D represents the length, and an encoder of the vision transformer model is composed of L consecutive layers of multi-head self-attention modules. The output of each layer is denoted as tₗ, l∈{1, . . . , L}.

The distribution and scale of skin lesions in clinical dermatological pictures vary greatly in space. Therefore, it is necessary for the model to have the ability to locate and capture a site with a nidus, so as to describe its attribute and accurately associate with a disease type. However, the token sequence generated by the vision transformer model is spatially redundant for the task performed, and therefore, a lesion token selection module, which may also be referred to as a nidus token selection module, is designed to guide the encoder of the vision transformer to select localized tokens associated with the niduses at different levels. Lesion token selection module 804 is configured to first calculate an attention matrix for each attention head in all multi-head self-attention modules:

$${A}^{m} = softmax ( \frac{Q {K}^{T}}{\sqrt{D}} ) ∈ {R}^{( {N}_{p} + 1 ) \times ( {N}_{p} + 1 )} , m ∈ \{ 1 , 2 , … , {N}_{h} \} \qquad ( 2 )$$

Then, normalized exponential functions (softmax) for the first row and first column of matrix Am are calculated as:

$${a}_{0 , n}^{m} = \frac{{e}^{{A}_{0 n}^{m}}}{{\sum }_{j = 1}^{{N}_{p}} {e}^{{A}_{0 j}^{m}}} , and {a}_{n , 0}^{m} = \frac{{e}^{{A}_{n , 0}^{m}}}{{\sum }_{j = 1}^{{N}_{p}} {e}^{{A}_{j , 0}^{m}}} , n ∈ \{ 0 , 1 , … , {N}_{p} \} \qquad ( 3 )$$

The above formulas (2) and (3) can express a score of the attention between a class token and another spatial token, calculate mutual attention scores for all heads, finally perform sorting based on importance levels of local tokens, and select first K tokens from the I-th layer. Nh represents the quantity of attention heads, and Q (Query) and K (Key) represent inputs of the attention modules, respectively.

The determination of a disease type requires not only fine-grained local features, but also global context information, such as a body part where the nidus appears. Therefore, it is necessary to learn an interaction relationship between a class token and a selected local token, that is, the fusion between global and local semantic information. Disease attribute joint learning module is configured to fuse features by using the selected local token and a class token of the last transformer layer.

First, two additional fusion modules are introduced. Two classification heads are defined for given samples, and outputs of the two introduced additional fusion modules are mapped to p_{c}^{k}∈Rⁿ^{_{c}}^{×1} and pₐ^{k} ∈Rⁿ^{ₐ}^{×1}. The two defined classification heads are substantially two fully connected layers (FCs). The two introduced additional fusion modules may be a contextual fusion module and a local fusion module. n_{c} and nₐ represent the quantities of conditions and attribute classes, respectively.

Next, a joint optimization objective function is defined as:

$${L}_{j oint} = - \frac{1}{{N}_{c}} {\sum }_{k = 1}^{{N}_{s}} {1}_{c} {\sum }_{i = 1}^{{n}_{c}} \frac{1 - β}{1 - {β}^{{n}_{ci}}} {y}_{c}^{k i} log ( {p}_{c}^{k i} ) - \frac{1}{{N}_{a}} {\sum }_{k = 1}^{{N}_{s}} {1}_{a} {\sum }_{j = 1}^{{n}_{a}} {y}_{a}^{k j} log ( {p}_{a}^{k j} ) \qquad ( 4 )$$

In the above formula (4), N_{c} represents the quantity of samples marked for diseases, Na represents the quantity of samples marked for attributes, β represents the inverse of the quantity of valid samples of the class, n_{c}_{ᵢ} represents the actual quantity of samples, y_{c}^{k} represents the one-hot encoded label of the disease category, yₐ^{k} represents the multi-hot nidus attribute label, 1_{c}/1ₐ represents an indicator function of the condition/attribute, and when the condition/attribute label is available, 1_{c}/1ₐ is 1, otherwise, 1_{c}/1ₐ is 0.

Finally, the problem of class imbalance is addressed based on a class balance loss.

In addition to performing a supervised training on the head of disease type and lesion attributes, the prior knowledge of dermatologists may further be used for calibrating the system in a case of misjudgment. Bilateral prediction distillation module 808 is configured to, based on statistics of diseases and attributes, calculate posterior distribution of the diseases given lesion attributes:

M(c|a)∈Rⁿ^{_{c}}^{×n}^{ₐ} (5)

The posterior distribution of the attributes is also calculated given a disease diagnosis:

M(aκ)∈Rⁿ^{ₐ}^{×n}^{_{c}} (6)

Each entry of the two matrices may be calculated as follows:

$$M ( {c}_{i} \vert {a}_{j} ) = \frac{{\sum }_{k} 1 [ {y}_{c}^{ki} \neq 0 , {y}_{a}^{k j} \neq 0 ]}{{\sum }_{k} 1 ⌈ {y}_{a}^{k j} \neq 0 ]} , \\ M ( {a}_{j} \vert {c}_{i} ) = \frac{{\sum }_{k} 1 [ {y}_{c}^{ki} \neq 0 , {y}_{a}^{k j} \neq 0 ]}{{\sum }_{k} 1 [ {y}_{c}^{ki} \neq 0 ]} \qquad ( 7 )$$

In the above formula (7), 1[·] represents an indicator function, and k represents a sample index.

For a given sample k, two probabilities p̅_̅{̅c̅}̅^̅{̅k̅}̅=M(c|a)pₐ^{k} and p̅ₐ̅^̅{̅k̅}̅=M(a|c)p_{c}^{k} of simultaneous occurrence are calculated, and a relative entropy (Kullback-Leibler divergence, KL divergence) between the two probabilities is obtained. The KL divergence may be used for representing asymmetric measurement of a difference between two probability distributions.

Different diseases may share the same attribute, and therefore, the distribution of p̅_̅{̅c̅}̅ is multimodal. However, the actual diagnosis is giving a unique and most likely result. That is, the distribution of p_{c} may be unimodal. Therefore, the precise matching between p̅_̅{̅c̅}̅ and p_{c} may be relaxed to a consistency measure.

Two consistency losses may be defined for the attribute and the disease type, respectively:

$$f ( {p}_{c}^{k} , \overline{{p}_{c}^{k}} ) = exp ( 1 - {p}_{c}^{k} \cdot \overline{{p}_{c}^{k}} ) - 1 \qquad ( 8 )$$

$${L}_{cond \_ attr} = \frac{1}{{N}_{a}} {\sum }_{k = 1}^{{N}_{s}} {1}_{a} \overline{{p}_{a}^{k}} ( log ( \overline{{p}_{a}^{k}} ) - log ( {p}_{a}^{k} ) ) \qquad ( 9 )$$

$${L}_{attr \_ cond} = - \frac{1}{{N}_{c}} {\sum }_{k = 1}^{{N}_{s}} {1}_{c} f ( {P}_{c}^{k} , \overline{{p}_{c}^{k}} ) {\sum }_{i = 1}^{{n}_{c}} \frac{1 - β}{1 - {β}^{{n}_{y c}}} {y}_{c}^{k i} log ( {p}_{c}^{k i} ) \qquad ( 10 )$$

The obtained loss function at the end includes three parts, which is:

Lₜₒₜₐₗ=Lⱼₒᵢₙₜ+a₁L_{cond_attr}+a₂L_{attr_cond} (11)

In the embodiments of the present disclosure, four different modules are used for processing the image information of the acquired skin image to extract the token sequence of the skin lesion region in the image of the skin. After the global information and the significant local information are fused, the loss function of attributes and disease types can be obtained through the calculation of the posterior distribution, so as to achieve the objective of accurately determining the pathological result, thereby achieving the technical effect of improving the accuracy of skin image detection. Furthermore, the image detection method provided by the embodiments of the present disclosure solves the technical problem of a low accuracy of skin image detection due to the complexity of the skin lesion attribute.

From the description of the above implementations, those skilled in the art may clearly understand that the method of skin detection for an object according to the above embodiments may be implemented by means of software plus a necessary general hardware platform, and may also be implemented by hardware. Based on the above understanding, the technical solutions of the present disclosure essentially, or the part making contributions to the prior art, can be embodied in the form of a software product. The computer software product is stored in a storage medium (such as a ROM/RAM, a magnetic disk, or an optical disc), and includes several instructions used for causing a terminal device (which may be a mobile terminal, a computer, a server, a network device, or the like) to perform the methods in various embodiments of the present disclosure.

According to some embodiments of the present disclosure, an apparatus for skin detection for an object for implementing the above method of skin detection for an object shown in FIG. 3 is further provided.

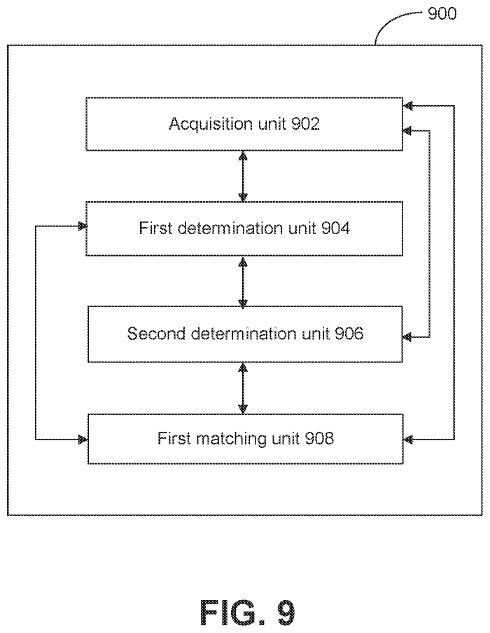

FIG. 9 is a schematic diagram of an apparatus for skin detection for an object according to some embodiments of the present disclosure. As shown in FIG. 9, the apparatus 900 for skin detection for an object may include an acquisition unit 902, a first determination unit 904, a second determination unit 906, and a first matching unit 908. It is appreciated that these units/modules may be hardware components or software components stored in memory (e.g., memory 104) for processing by one or more processors (e.g., processors 102a, 102b, 102n).

Acquisition unit 902 is configured to acquire an image of skin covering an outer surface of an object to be detected.

First determination unit 904 is configured to recognize the image of the skin, and determine a skin lesion region of the object to be detected. The skin lesion region is an image region with a skin lesion feature in the image of the skin.

Second determination unit 906 is configured to determine a skin lesion attribute of the object to be detected based on the skin lesion feature of the skin lesion region and an object type of the object to be detected. The skin lesion attribute is used for describing a skin lesion generated on the object to be detected.

First matching unit 908 is configured to match lesion data recorded in a lesion database based on the skin lesion attribute to determine a pathological result of the object to be detected.

It should be noted here that the above first acquisition unit 902, first determination unit 904, second determination unit 906, and first matching unit 908 correspond to steps S302 to S308 in the above embodiments. Examples and application scenarios implemented by the four units are the same as those of corresponding steps, but are not limited to the content disclosed in the above embodiments. It should be noted that, as a part of the apparatus, the above units may run in the AR/VR device provided in the above embodiments.

According to some embodiments of the present disclosure, an apparatus for skin detection for an object for implementing the above method of skin detection for an object shown in FIG. 4 is further provided.

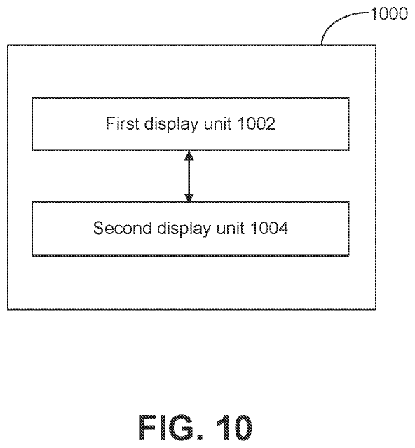

FIG. 10 is a schematic diagram of an apparatus for skin detection for an object according to some embodiments of the present disclosure. As shown in FIG. 10, the apparatus 1000 for skin detection for an object may include a first display unit 1002 and a second display unit 1004 (e.g., the display shown in FIG. 1). It is appreciated that these units/modules may be hardware components controlled by one or more processors (e.g., processors 102a, 102b, 102n). In some embodiments, the first display unit 1002 and the second display unit 1004 may also be different parts of the same display (e.g., the display shown in FIG. 1), but the present disclosure is not limited thereto.

First display unit 1002 is configured to display, in response to an image input instruction acting on an operation interface, an image of skin covering an outer surface of an object to be detected from a medical diagnosis platform on the operation interface.

Second display unit 1004 is configured to display, in response to a detection operation instruction acting on the operation interface, a pathological result of the object to be detected on the operation interface. The pathological result is obtained by matching lesion data recorded in a lesion database based on a skin lesion attribute of the object to be detected. The skin lesion attribute is determined based on a skin lesion feature of a skin lesion region and an object type of the object to be detected. The skin lesion region is obtained by recognizing the image of the skin.

It should be noted here that the above first display unit 1002 and second display unit 1004 correspond to steps S402 to S404 in the above embodiments. Examples and application scenarios implemented by the two units are the same as those of corresponding steps, but are not limited to the content disclosed in the above embodiments. It should be noted that, as a part of the apparatus, the above units may run in the AR/VR device provided in the above embodiments.

According to some embodiments of the present disclosure, an apparatus for skin detection for an object for implementing the above method of skin detection for an object shown in FIG. 5 is further provided.

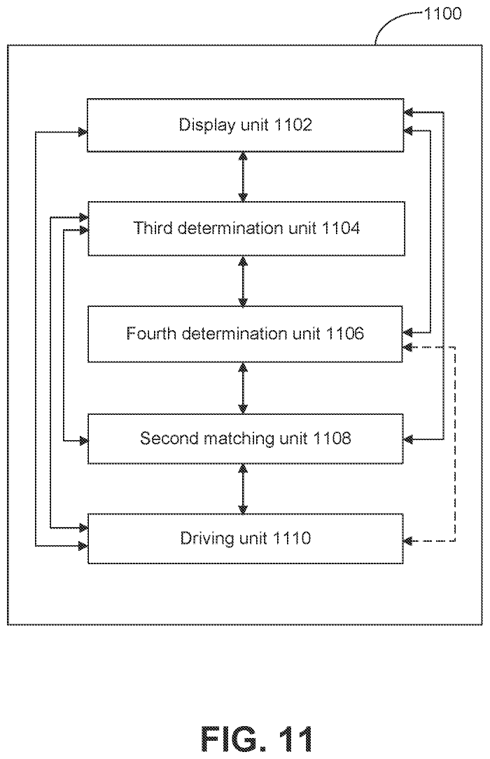

FIG. 11 is a schematic diagram of an apparatus for skin detection for an object according to some embodiments of the present disclosure. As shown in FIG. 11, the apparatus 1100 for skin detection for an object may include a display unit 1102 (e.g., display in FIG. 1), a third determination unit 1104, a fourth determination unit 1106, a second matching unit 1108, and a driving unit 1110. It is appreciated that these units/modules may be hardware components or software components stored in memory (e.g., memory 104) for processing by one or more processors (e.g., processors 102a, 102b, 102n).

Display unit 1102 is configured to display an image of skin covering an outer surface of an object to be detected on a presentation screen of a virtual reality (VR) device or an augmented reality (AR) device.

Third determination unit 1104 is configured to recognize the image of the skin, and determine a skin lesion region of the object to be detected. The skin lesion region is an image region with a skin lesion feature in the image of the skin sensed by the VR device or AR device.

Fourth determination unit 1106 is configured to determine a skin lesion attribute of the object to be detected based on the skin lesion feature of the skin lesion region and an object type of the object to be detected. The skin lesion attribute is used for describing a skin lesion generated on the object to be detected.

Second matching unit 1108 is configured to match lesion data recorded in a lesion database based on the skin lesion attribute to determine a pathological result of the object to be detected.

Driving unit 1110 is configured to drive the VR device or AR device to display the skin lesion attribute and the pathological result.

It should be noted here that the above display unit 1102, third determination unit 1104, fourth determination unit 1106, second matching unit 1108, and driving unit 1110 correspond to steps S502 to S510 in the above embodiments. Examples and application scenarios implemented by the five units are the same as those of corresponding steps, but are not limited to the content disclosed in the above embodiments. It should be noted that, as a part of the apparatus, the above units may run in the AR/VR device provided in the above embodiments.

According to some embodiments of the present disclosure, an apparatus for skin detection for an object for implementing the above method of skin detection for an object shown in FIG. 6 is further provided.

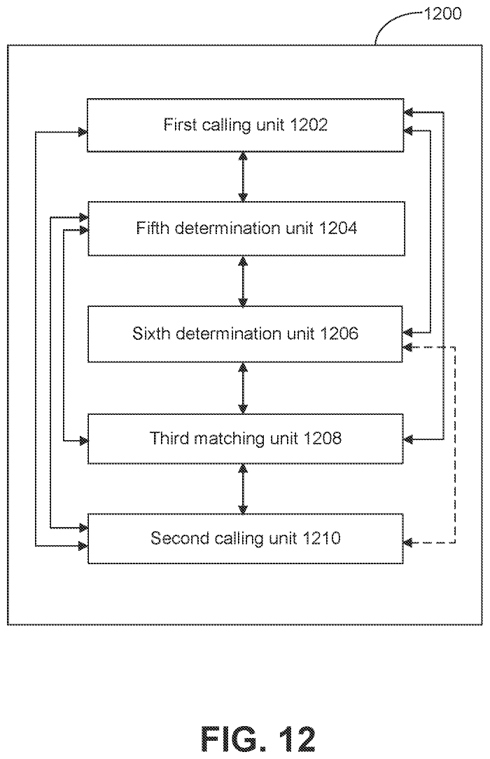

FIG. 12 is a schematic diagram of an apparatus for skin detection for an object according to some embodiments of the present disclosure. As shown in FIG. 12, the apparatus 1200 for skin detection for an object may include a first calling unit 1202, a fifth determination unit 1204, a sixth determination unit 1206, a third matching unit 1208, and a second calling unit 1210. It is appreciated that these units/modules may be hardware components or software components stored in memory (e.g., memory 104) for processing by one or more processors (e.g., processors 102a, 102b, 102n).

First calling unit 1202 is configured to acquire, by calling a first interface, an image of skin covering an outer surface of an object to be detected. The first interface includes a first parameter, and a parameter value of the first parameter is the image of the skin.

Fifth determination unit 1204 is configured to recognize the image of the skin, and determine a skin lesion region of the object to be detected. The skin lesion region is an image region with a skin lesion feature in the image of the skin.

Sixth determination unit 1206 is configured to determine a skin lesion attribute of the object to be detected based on the skin lesion feature of the skin lesion region and an object type of the object to be detected. The skin lesion attribute is used for describing a skin lesion generated on the object to be detected.

Third matching unit 1208 is configured to match lesion data recorded in a lesion database based on the skin lesion attribute to determine a pathological result of the object to be detected.

Second calling unit 1210 is configured to output, by calling a second interface, the skin lesion attribute and the pathological result. The second interface includes a second parameter, and a value of the second parameter is the skin lesion attribute and the pathological result.

It should be noted here that the above first calling unit 1202, fifth determination unit 1204, sixth determination unit 1206, third matching unit 1208, and second calling unit 1210 correspond to steps S602 to S610 in the above embodiments. Examples and application scenarios implemented by the five units are the same as those of corresponding steps, but are not limited to the content disclosed in the above embodiments. It should be noted that, as a part of the apparatus, the above units may run in the AR/VR device provided in the above embodiments.

In the present embodiments, by determining the skin lesion disease region, matching the skin lesion attribute of the skin lesion region with the lesion data recorded in the lesion database, determining an attribute feature of a disease, and displaying the skin lesion attribute and pathological result on the related interface, an objective of accurately determining the pathological result is achieved, thereby achieving the technical effect of improving the detection accuracy of the image of the skin of the object. Furthermore, the image detection method provided by the embodiments of the present disclosure solves the problem of a low detection accuracy for an image of skin of an object.

The embodiments of the present disclosure may provide a computer terminal. The computer terminal may be any computer terminal device in a computer terminal group. Optionally, in the present embodiments, the above computer terminal may also be replaced by a terminal device such as a mobile terminal.

Optionally, in the present embodiments, the above computer terminal may be located in at least one network device among multiple network devices of a computer network.

In the present embodiments, the above computer terminal may execute program codes of the following steps in the method of skin detection for an object: acquiring an image of skin covering an outer surface of an object to be detected; evaluating the image of the skin to determine a skin lesion region of the object to be detected, the skin lesion region being an image region with a skin lesion feature in the image of the skin; determining a skin lesion attribute of the object to be detected based on the skin lesion feature of the skin lesion region and an object type of the object to be detected, the skin lesion attribute being used for describing a skin lesion generated on the object to be detected; and matching lesion data recorded in a lesion database based on the skin lesion attribute to determine a pathological result of the object to be detected.

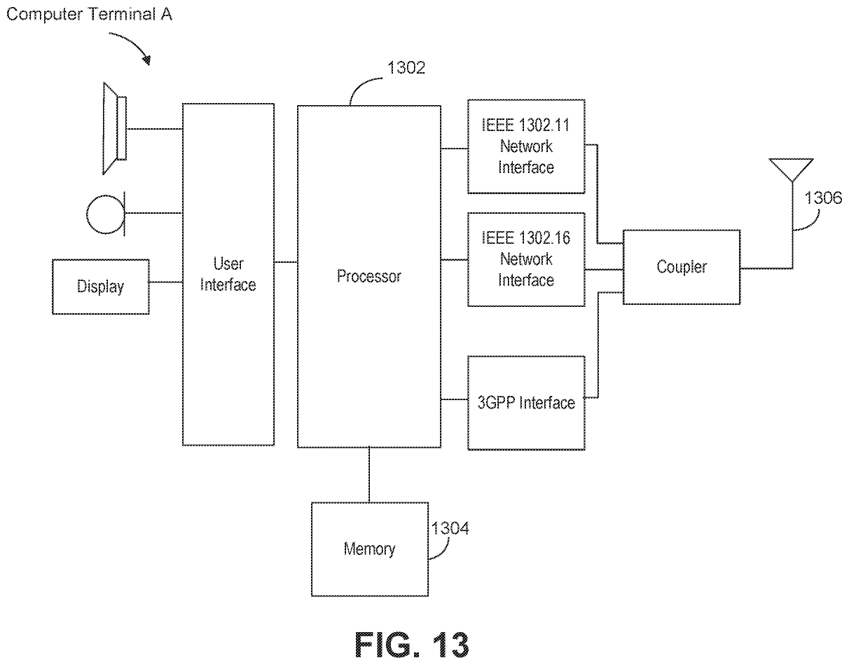

Optionally, FIG. 13 is a structural block diagram of a computer terminal according to some embodiments of the present disclosure. As shown in FIG. 13, the computer terminal A may include one or multiple (only one shown in the figure) processors 1302, a memory 1304, and a transmission apparatus 1306.

The memory may be configured to store software programs and modules, such as program instructions/modules corresponding to the method and apparatus for skin detection for an object in the embodiments of the present disclosure. The processor performs various functional applications and data processing, that is, implements the above method of skin detection for an object, by running the software programs and modules stored in the memory. The memory may include a high-speed random access memory, and may further include a non-volatile memory, such as one or multiple magnetic storage devices, a flash memory, or another non-volatile solid-state memory. In some examples, the memory may further include memories arranged remotely with respect to the processor, and these remote memories may be connected to computer terminal A through a network. Examples of the above network include, but are not limited to, the Internet, an intranet, a local area network, a mobile communication network, and any combination thereof.

The processor may perform the following steps by calling, via the transmission apparatus, information and application programs stored in the memory: acquiring an image of skin covering an outer surface of an object to be detected; evaluating the image of the skin to determine a skin lesion region of the object to be detected, the skin lesion region being an image region with a skin lesion feature in the image of the skin; determining a skin lesion attribute of the object to be detected based on the skin lesion feature of the skin lesion region and an object type of the object to be detected, the skin lesion attribute being used for describing a skin lesion generated on the object to be detected; and matching lesion data recorded in a lesion database based on the skin lesion attribute to determine a pathological result of the object to be detected.

Optionally, the above processor may further execute the program code for the following steps: recognizing the image of the skin to obtain a region token sequence, each region token in the region token sequence being used for representing a skin region of the object to be detected; and extracting a sub-region token sequence from the region token sequence, each region token in the sub-region token sequence being used for representing a sub-skin lesion region in the skin lesion region.

Optionally, the above processor may further execute the program code for the following steps: converting the region token sequence into an image feature sequence of the image of the skin, an image feature in the image feature sequence being used for representing the skin region corresponding to the region token in the region token sequence; determining a sub-image feature sequence corresponding to the skin lesion region from the image feature sequence, the skin lesion region including the skin region corresponding to the image feature in the sub-image feature sequence; and determining the sub-region token sequence corresponding to the sub-image feature sequence.

Optionally, the above processor may further execute the program code for the following step: determining at least one region token having an importance level higher than a target threshold in the region token sequence as the sub-region token sequence, the importance level being used for representing a level of importance of the corresponding region token to the pathological result.

Optionally, the above processor may further execute the program code for the following step: selecting the at least one region token having the importance level higher than the target threshold from the region token sequence based on a lesion token selection module, the lesion token selection module being at least configured to determine the importance level.

Optionally, the above processor may further execute the program code for the following step: partitioning the image of the skin into multiple region tokens based on a vision transformer model to obtain the region token sequence, the vision transformer model being obtained by training based on a self-attention mechanism.

Optionally, the above processor may further execute the program code for the following step: fusing the skin lesion features of the multiple sub-skin lesion regions corresponding to the sub-region token sequence and the object type to obtain the skin lesion attribute.

Optionally, the above processor may further execute the program code for the following step: calibrating the skin lesion attribute based on a target skin lesion attribute of the object to be detected, or calibrating the pathological result based on a target pathological result of the object to be detected, so that the skin lesion attribute matches the pathological result.

In some embodiments, the processor may perform the following steps by calling, via the transmission apparatus, the information and application programs stored in the memory: displaying, in response to an image input instruction acting on an operation interface, an image of skin covering an outer surface of an object to be detected from a medical diagnosis platform on the operation interface; and displaying, in response to a detection operation instruction acting on the operation interface, a pathological result of the object to be detected on the operation interface. The pathological result is obtained by matching lesion data recorded in a lesion database based on a skin lesion attribute of the object to be detected. The skin lesion attribute is determined based on a skin lesion feature of a skin lesion region and an object type of the object to be detected. The skin lesion region is obtained by recognizing the image of the skin.

In some embodiments, the processor may perform the following steps by calling, via the transmission apparatus, the information and application programs stored in the memory: displaying an image of skin covering an outer surface of an object to be detected on a presentation screen of a virtual reality (VR) device or an augmented reality (AR) device; evaluating the image of the skin to determine a skin lesion region of the object to be detected, the skin lesion region being an image region with a skin lesion feature in the image of the skin sensed by the VR device or the AR device; determining a skin lesion attribute of the object to be detected based on the skin lesion feature of the skin lesion region and an object type of the object to be detected, the skin lesion attribute being used for describing a skin lesion generated on the object to be detected; matching lesion data recorded in a lesion database based on the skin lesion attribute to determine a pathological result of the object to be detected; and driving the VR device or the AR device to display the skin lesion attribute and the pathological result.

In some embodiments, the processor may perform the following steps by calling, via the transmission apparatus, the information and application programs stored in the memory: acquiring, by calling a first interface, an image of skin covering an outer surface of an object to be detected, the first interface including a first parameter, and a parameter value of the first parameter being the image of the skin; evaluating the image of the skin to determine a skin lesion region of the object to be detected, the skin lesion region is an image region with a skin lesion feature in the image of the skin; determining a skin lesion attribute of the object to be detected based on the skin lesion feature of the skin lesion region and an object type of the object to be detected, the skin lesion attribute being used for describing a skin lesion generated on the object to be detected; matching lesion data recorded in a lesion database based on the skin lesion attribute to determine a pathological result of the object to be detected; and outputting, by calling a second interface, the skin lesion attribute and the pathological result, the second interface including a second parameter, and a value of the second parameter being the skin lesion attribute and the pathological result.

The embodiments of the present disclosure provide a method of skin detection for an object, and by paying attention to the skin lesion disease region, matching the skin lesion attribute of the determined skin lesion region with the lesion data recorded in the lesion database, and determining an attribute feature of a disease, an objective of accurately determining the pathological result is achieved, thereby achieving the technical effect of improving the detection accuracy of the image of the skin of the object. Furthermore, the image detection method provided by the embodiments of the present disclosure solves the technical problem of a low detection accuracy for an image of skin of an object.

Those of ordinary skill in the art can understand that the structure shown in FIG. 13 is merely illustrative, and the computer terminal may also be a smart phone (e.g., an Android phone and an iOS phone), a tablet computer, a palmtop computer, and a Mobile Internet Device (MID), a PAD, or another terminal device. FIG. 13 does not limit the structure of the above electronic apparatus. For example, computer terminal A may also include more or fewer components (such as a network interface and a display apparatus) than that shown in FIG. 13, or have a different configuration from that shown in FIG. 13.

Those of ordinary skill in the art can understand that all or part of the steps in the various methods of the above embodiments may be completed by instructing the hardware related to the terminal device through a program. The program may be stored in a computer-readable storage medium, and the storage medium may include: a flash disk, a Read-Only Memory (ROM), a Random Access Memory (RAM), a magnetic disk, an optical disc, or the like.

A computer-readable storage medium is further provided in the embodiments of the present disclosure. Optionally, in the present embodiments, the above computer-readable storage medium may be configured to store the program codes executed by the method of skin detection for an object provided in the above embodiments.

Optionally, in the present embodiments, the computer-readable storage medium may be located in any computer terminal in a computer terminal group in a computer network, or in any mobile terminal in a mobile terminal group.

Optionally, in the embodiments of the present disclosure, the computer-readable storage medium is configured to store program codes for performing the following steps: acquiring an image of skin covering an outer surface of an object to be detected; evaluating the image of the skin to determine a skin lesion region of the object to be detected, the skin lesion region being an image region with a skin lesion feature in the image of the skin; determining a skin lesion attribute of the object to be detected based on the skin lesion feature of the skin lesion region and an object type of the object to be detected, the skin lesion attribute being used for describing a skin lesion generated on the object to be detected; and matching lesion data recorded in a lesion database based on the skin lesion attribute to determine a pathological result of the object to be detected.

Optionally, the above computer-readable storage medium may further execute program codes for the following steps: recognizing the image of the skin to obtain a region token sequence, each region token in the region token sequence being used for representing a skin region of the object to be detected; and extracting a sub-region token sequence from the region token sequence, each region token in the sub-region token sequence being used for representing a sub-skin lesion region in the skin lesion region.

Optionally, the above computer-readable storage medium may further execute program codes for the following steps: converting the region token sequence into an image feature sequence of the image of the skin, an image feature in the image feature sequence being used for representing the skin region corresponding to the region token in the region token sequence; determining a sub-image feature sequence corresponding to the skin lesion region from the image feature sequence, the skin lesion region including the skin region corresponding to the image feature in the sub-image feature sequence; and determining the sub-region token sequence corresponding to the sub-image feature sequence.

Optionally, the above computer-readable storage medium may further execute program codes for the following step: determining at least one region token having an importance level higher than a target threshold in the region token sequence as the sub-region token sequence, the importance level being used for representing a level of importance of the corresponding region token to the pathological result.

Optionally, the above computer-readable storage medium may further execute program codes for the following step: selecting the at least one region token having the importance level higher than the target threshold from the region token sequence based on a lesion token selection module, the lesion token selection module being at least configured to determine the importance level.

Optionally, the above computer-readable storage medium may further execute program codes for the following step: partitioning the image of the skin into multiple the region tokens based on a vision transformer model to obtain the region token sequence, the vision transformer model being obtained by training based on a self-attention mechanism.

Optionally, the above computer-readable storage medium may further execute program codes for the following step: fusing the skin lesion features of the multiple sub-skin lesion regions corresponding to the sub-region token sequence and the object type to obtain the skin lesion attribute.

Optionally, the above computer-readable storage medium may further execute program codes for the following step: calibrating the skin lesion attribute based on a target skin lesion attribute of the object to be detected, or calibrating the pathological result based on a target pathological result of the object to be detected, so that the skin lesion attribute matches the pathological result.

In some embodiments, the computer-readable storage medium may be configured to store program codes for performing the following steps: displaying, in response to an image input instruction acting on an operation interface, an image of skin covering an outer surface of an object to be detected from a medical diagnosis platform on the operation interface; and displaying, in response to a detection operation instruction acting on the operation interface, a pathological result of the object to be detected on the operation interface. The pathological result is obtained by matching lesion data recorded in a lesion database based on a skin lesion attribute of the object to be detected. The skin lesion attribute is determined based on a skin lesion feature of a skin lesion region and an object type of the object to be detected. The skin lesion region is obtained by recognizing the image of the skin.

In some embodiments, the computer-readable storage medium may be configured to store program codes for performing the following steps: displaying an image of skin covering an outer surface of an object to be detected on a presentation screen of a virtual reality (VR) device or an augmented reality (AR) device; evaluating the image of the skin to determine a skin lesion region of the object to be detected, the skin lesion region being an image region with a skin lesion feature in the image of the skin sensed by the VR device or the AR device; determining a skin lesion attribute of the object to be detected based on the skin lesion feature of the skin lesion region and an object type of the object to be detected, the skin lesion attribute being used for describing a skin lesion generated on the object to be detected; matching lesion data recorded in a lesion database based on the skin lesion attribute to determine a pathological result of the object to be detected; and driving the VR device or the AR device to display the skin lesion attribute and the pathological result.

In some embodiments, the computer-readable storage medium may be configured to store program codes for performing the following steps: acquiring, by calling a first interface, an image of skin covering an outer surface of an object to be detected, the first interface including a first parameter, and a parameter value of the first parameter being the image of the skin; evaluating the image of the skin to determine a skin lesion region of the object to be detected, the skin lesion region being an image region with a skin lesion feature in the image of the skin; determining a skin lesion attribute of the object to be detected based on the skin lesion feature of the skin lesion region and an object type of the object to be detected, the skin lesion attribute being used for describing a skin lesion generated on the object to be detected; matching lesion data recorded in a lesion database based on the skin lesion attribute to determine a pathological result of the object to be detected; and outputting, by calling a second interface, the skin lesion attribute and the pathological result, the second interface including a second parameter, and a value of the second parameter being the skin lesion attribute and the pathological result.

The above sequence of the embodiments of the present disclosure is only for the ease of description, and does not represent that one embodiment is better than another.

In the above embodiments of the present disclosure, the description of each embodiments has its own emphasis. For parts that are not described in detail in a certain embodiment, reference may be made to related descriptions of other embodiments.

In the various embodiments provided in the present disclosure, it should be understood that the disclosed technical content may be implemented in other manners. The apparatus embodiments described above are only examples. For example, the division of the units is only a logical function division. In actual implementations, there may be another division manner. For example, multiple units or components may be combined or integrated into another system, or some features can be omitted or not implemented. In addition, the displayed or discussed mutual coupling or direct coupling or communicative connection may be indirect coupling or communicative connection through some interfaces, units, or modules, and may be in electrical or other forms.

The units described as separate components may or may not be physically separated, and the components displayed as units may or may not be physical units, that is, they may be located in one place, or may be distributed to multiple network units. Part or all of the units may be selected according to actual needs to achieve the purpose of the solution of the present embodiments.

In addition, functional units in the embodiments of the present disclosure may be integrated into one processing unit, or each unit may exist physically alone, or two or more units may be integrated into one unit. The above integrated unit may be implemented in the form of hardware, or may be implemented in the form of a software functional unit.

The integrated unit, when implemented in the form of a software functional unit and sold or used as an independent product, may be stored in a computer-readable storage medium. Based on the above understanding, the technical solutions of the present specification essentially, or the part making contributions to the prior art, or all or part of the technical solution, may be embodied in the form of a software product. The computer software product may be stored in a storage medium, and includes several instructions configured to cause a computer device (which may be a personal computer, a server, a network device, or the like) to perform all or part of the steps of the methods in various embodiments of the present disclosure. The aforementioned storage medium includes: a USB flash drive, a Read-Only Memory (ROM), a Random Access Memory (RAM), a mobile hard disk drive, a magnetic disk, an optical disc, or other media that can store program codes.

The embodiments may further be described using the following clauses:

1. A method of skin detection for an object, comprising:
acquiring an image of skin covering an outer surface of an object to be detected;
evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected; and
matching lesion data recorded in a lesion database based on the skin lesion attribute to determine a pathological result of the object to be detected.

2. The method of clause 1, wherein evaluating the image of the skin to determine the skin lesion region of the object to be detected comprises:
recognizing the image of the skin to obtain a region token sequence, wherein each region token in the region token sequence is used for representing a skin region of the object to be detected; and
extracting a sub-region token sequence from the region token sequence, wherein each region token in the sub-region token sequence is used for representing a sub-skin lesion region in the skin lesion region.

3. The method of clause 2, wherein extracting the sub-region token sequence from the region token sequence comprises:
converting the region token sequence into an image feature sequence of the image of the skin, wherein an image feature in the image feature sequence is used for representing the skin region corresponding to the region token in the region token sequence;
determining a sub-image feature sequence corresponding to the skin lesion region from the image feature sequence, wherein the skin lesion region comprises the skin region corresponding to the image feature in the sub-image feature sequence; and
determining the sub-region token sequence corresponding to the sub-image feature sequence.

4. The method of clause 2 or 3, wherein extracting the sub-region token sequence from the region token sequence comprises:
determining at least one region token having an importance level higher than a target threshold in the region token sequence as the sub-region token sequence, wherein the importance level is used for representing a level of importance of the corresponding region token to the pathological result.

5. The method of clause 4, wherein determining the at least one region token having the importance level higher than the target threshold in the region token sequence as the sub-region token sequence comprises:
selecting the at least one region token having the importance level higher than the target threshold from the region token sequence based on a lesion token selection module, wherein the lesion token selection module is at least configured to determine the importance level.

6. The method of any of clauses 2-5, wherein generating the region token sequence for the image of the skin comprises:
partitioning the image of the skin into a plurality of the region tokens based on a vision transformer model to obtain the region token sequence, wherein the vision transformer model is obtained by training based on a self-attention mechanism.

7. The method of any of clauses 2-6, wherein determining the skin lesion attribute of the object to be detected based on the skin lesion feature of the skin lesion region and the object type of the object to be detected comprises:
fusing the skin lesion features of the plurality of sub-skin lesion regions corresponding to the sub-region token sequence with the object type to obtain the skin lesion attribute.

8. The method of any of clauses 1-7, further comprising:
calibrating the skin lesion attribute based on a target skin lesion attribute of the object to be detected, or calibrating the pathological result based on a target pathological result of the object to be detected, in order to match the skin lesion attribute with the pathological result.

9. A method of skin detection for an object, comprising:
displaying, in response to an image input instruction acting on an operation interface, an image of skin covering an outer surface of an object to be detected from a medical diagnosis platform on the operation interface; and
displaying, in response to a detection operation instruction acting on the operation interface, a pathological result of the object to be detected on the operation interface, wherein the pathological result is obtained by matching lesion data recorded in a lesion database based on a skin lesion attribute of the object to be detected, the skin lesion attribute is determined based on a skin lesion feature of a skin lesion region and an object type of the object to be detected, and the skin lesion region is obtained by recognizing the image of the skin.

10. A method of skin detection for an object, comprising:
displaying an image of skin covering an outer surface of an object to be detected on a presentation screen of a virtual reality (VR) device or an augmented reality (AR) device;
evaluating the image of the skin to determine a skin lesion region of the object to be detected, wherein the skin lesion region is an image region with a skin lesion feature in the image of the skin sensed by the VR device or the AR device;
determining a skin lesion attribute of the object to be detected based on the skin lesion feature of the skin lesion region and an object type of the object to be detected, wherein the skin lesion attribute is used for describing a skin lesion generated on the object to be detected;
matching lesion data recorded in a lesion database based on the skin lesion attribute to determine a pathological result of the object to be detected; and driving the VR device or the AR device to display the skin lesion attribute and the pathological result.

11. A method of skin detection for an object, comprising:
acquiring, by calling a first interface, an image of skin covering an outer surface of an object to be detected, wherein the first interface comprises a first parameter, and a parameter value of the first parameter is the image of the skin;
evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected;
matching lesion data recorded in a lesion database based on the skin lesion attribute to determine a pathological result of the object to be detected; and
outputting, by calling a second interface, the skin lesion attribute and the pathological result, wherein the second interface comprises a second parameter, and a value of the second parameter is the skin lesion attribute and the pathological result.

12. A non-transitory computer-readable medium storing a set of instructions that is executable by at least one processor of an apparatus to cause the apparatus to perform a method of skin detection comprising:
acquiring an image of skin covering an outer surface of an object to be detected;
evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected; and
matching lesion data recorded in a lesion database based on the skin lesion attribute to determine a pathological result of the object to be detected.

13. The computer-readable storage medium of clause 12, wherein evaluating the image of the skin to determine the skin lesion region of the object to be detected comprises:
recognizing the image of the skin to obtain a region token sequence, wherein each region token in the region token sequence is used for representing a skin region of the object to be detected; and
extracting a sub-region token sequence from the region token sequence, wherein each region token in the sub-region token sequence is used for representing a sub-skin lesion region in the skin lesion region.

14. The computer-readable storage medium of clause 13, wherein extracting the sub-region token sequence from the region token sequence comprises:
converting the region token sequence into an image feature sequence of the image of the skin, wherein an image feature in the image feature sequence is used for representing the skin region corresponding to the region token in the region token sequence;
determining a sub-image feature sequence corresponding to the skin lesion region from the image feature sequence, wherein the skin lesion region comprises the skin region corresponding to the image feature in the sub-image feature sequence; and
determining the sub-region token sequence corresponding to the sub-image feature sequence.

15. The computer-readable storage medium of clause 13 or 14, wherein extracting the sub-region token sequence from the region token sequence comprises:
determining at least one region token having an importance level higher than a target threshold in the region token sequence as the sub-region token sequence, wherein the importance level is used for representing a level of importance of the corresponding region token to the pathological result.

16. The computer-readable storage medium of clause 15, wherein determining the at least one region token having the importance level higher than the target threshold in the region token sequence as the sub-region token sequence comprises:
selecting the at least one region token having the importance level higher than the target threshold from the region token sequence based on a lesion token selection module, wherein the lesion token selection module is at least configured to determine the importance level.

17. The computer-readable storage medium of any of clause 13-16, wherein generating the region token sequence for the image of the skin comprises:
partitioning the image of the skin into a plurality of the region tokens based on a vision transformer model to obtain the region token sequence, wherein the vision transformer model is obtained by training based on a self-attention mechanism.

18. The computer-readable storage medium of any of clauses 13-17, wherein determining the skin lesion attribute of the object to be detected based on the skin lesion feature of the skin lesion region and the object type of the object to be detected comprises:
fusing the skin lesion features of the plurality of sub-skin lesion regions corresponding to the sub-region token sequence with the object type to obtain the skin lesion attribute.

19. The computer-readable storage medium of any of clause 12-18, wherein the set of instructions is executable by the at least one processor of the apparatus to cause the apparatus to further perform:
calibrating the skin lesion attribute based on a target skin lesion attribute of the object to be detected, or calibrating the pathological result based on a target pathological result of the object to be detected, in order to match the skin lesion attribute with the pathological result.

20. A non-transitory computer-readable medium storing a set of instructions that is executable by at least one processor of an apparatus to cause the apparatus to perform a method of skin detection comprising:
displaying, in response to an image input instruction acting on an operation interface, an image of skin covering an outer surface of an object to be detected from a medical diagnosis platform on the operation interface; and
displaying, in response to a detection operation instruction acting on the operation interface, a pathological result of the object to be detected on the operation interface, wherein the pathological result is obtained by matching lesion data recorded in a lesion database based on a skin lesion attribute of the object to be detected, the skin lesion attribute is determined based on a skin lesion feature of a skin lesion region and an object type of the object to be detected, and the skin lesion region is obtained by recognizing the image of the skin.

21. A non-transitory computer-readable medium storing a set of instructions that is executable by at least one processor of an apparatus to cause the apparatus to perform a method of skin detection comprising:
displaying an image of skin covering an outer surface of an object to be detected on a presentation screen of a virtual reality (VR) device or an augmented reality (AR) device;
evaluating the image of the skin to determine a skin lesion region of the object to be detected, wherein the skin lesion region is an image region with a skin lesion feature in the image of the skin sensed by the VR device or the AR device;
determining a skin lesion attribute of the object to be detected based on the skin lesion feature of the skin lesion region and an object type of the object to be detected, wherein the skin lesion attribute is used for describing a skin lesion generated on the object to be detected;
matching lesion data recorded in a lesion database based on the skin lesion attribute to determine a pathological result of the object to be detected; and driving the VR device or the AR device to display the skin lesion attribute and the pathological result.

22. A non-transitory computer-readable medium storing a set of instructions that is executable by at least one processor of an apparatus to cause the apparatus to perform a method of skin detection comprising:
acquiring, by calling a first interface, an image of skin covering an outer surface of an object to be detected, wherein the first interface comprises a first parameter, and a parameter value of the first parameter is the image of the skin;
evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected;
matching lesion data recorded in a lesion database based on the skin lesion attribute to determine a pathological result of the object to be detected; and
outputting, by calling a second interface, the skin lesion attribute and the pathological result, wherein the second interface comprises a second parameter, and a value of the second parameter is the skin lesion attribute and the pathological result.

23. A system comprising:
a memory storing a set of instructions;
one or more processors configured to execute the set of instructions to cause the system to perform:
acquiring an image of skin covering an outer surface of an object to be detected; evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected; and
matching lesion data recorded in a lesion database based on the skin lesion attribute to determine a pathological result of the object to be detected.

24. The system of clause 23, wherein evaluating the image of the skin to determine the skin lesion region of the object to be detected comprises:
recognizing the image of the skin to obtain a region token sequence, wherein each region token in the region token sequence is used for representing a skin region of the object to be detected; and
extracting a sub-region token sequence from the region token sequence, wherein each region token in the sub-region token sequence is used for representing a sub-skin lesion region in the skin lesion region.

25. The system of clause 24, wherein extracting the sub-region token sequence from the region token sequence comprises:
converting the region token sequence into an image feature sequence of the image of the skin, wherein an image feature in the image feature sequence is used for representing the skin region corresponding to the region token in the region token sequence;
determining a sub-image feature sequence corresponding to the skin lesion region from the image feature sequence, wherein the skin lesion region comprises the skin region corresponding to the image feature in the sub-image feature sequence; and
determining the sub-region token sequence corresponding to the sub-image feature sequence.

26. The system of clause 24 or 25, wherein extracting the sub-region token sequence from the region token sequence comprises:
determining at least one region token having an importance level higher than a target threshold in the region token sequence as the sub-region token sequence, wherein the importance level is used for representing a level of importance of the corresponding region token to the pathological result.

27. The system of clause 26, wherein determining the at least one region token having the importance level higher than the target threshold in the region token sequence as the sub-region token sequence comprises:
selecting the at least one region token having the importance level higher than the target threshold from the region token sequence based on a lesion token selection module, wherein the lesion token selection module is at least configured to determine the importance level.

28. The system of any of clauses 24-27, wherein generating the region token sequence for the image of the skin comprises:
partitioning the image of the skin into a plurality of the region tokens based on a vision transformer model to obtain the region token sequence, wherein the vision transformer model is obtained by training based on a self-attention mechanism.

29. The system of any of clauses 24-28, wherein determining the skin lesion attribute of the object to be detected based on the skin lesion feature of the skin lesion region and the object type of the object to be detected comprises:
fusing the skin lesion features of the plurality of sub-skin lesion regions corresponding to the sub-region token sequence with the object type to obtain the skin lesion attribute.

30. The system of any of clause 23-29, wherein the one or more processors are configured to execute the set of instructions to cause the system to further perform:
calibrating the skin lesion attribute based on a target skin lesion attribute of the object to be detected, or calibrating the pathological result based on a target pathological result of the object to be detected, in order to match the skin lesion attribute with the pathological result.

31. A system comprising:
a memory storing a set of instructions;
one or more processors configured to execute the set of instructions to cause the system to perform:
   displaying, in response to an image input instruction acting on an operation interface, an image of skin covering an outer surface of an object to be detected from a medical diagnosis platform on the operation interface; and
   displaying, in response to a detection operation instruction acting on the operation interface, a pathological result of the object to be detected on the operation interface, wherein the pathological result is obtained by matching lesion data recorded in a lesion database based on a skin lesion attribute of the object to be detected, the skin lesion attribute is determined based on a skin lesion feature of a skin lesion region and an object type of the object to be detected, and the skin lesion region is obtained by recognizing the image of the skin.

32. A system comprising:
a memory storing a set of instructions;
one or more processors configured to execute the set of instructions to cause the system to perform:
displaying an image of skin covering an outer surface of an object to be detected on a presentation screen of a virtual reality (VR) device or an augmented reality (AR) device;
evaluating the image of the skin to determine a skin lesion region of the object to be detected, wherein the skin lesion region is an image region with a skin lesion feature in the image of the skin sensed by the VR device or the AR device;
determining a skin lesion attribute of the object to be detected based on the skin lesion feature of the skin lesion region and an object type of the object to be detected, wherein the skin lesion attribute is used for describing a skin lesion generated on the object to be detected;
matching lesion data recorded in a lesion database based on the skin lesion attribute to determine a pathological result of the object to be detected; and driving the VR device or the AR device to display the skin lesion attribute and the pathological result.

33. A system comprising:
a memory storing a set of instructions;
one or more processors configured to execute the set of instructions to cause the system to perform:
acquiring, by calling a first interface, an image of skin covering an outer surface of an object to be detected, wherein the first interface comprises a first parameter, and a parameter value of the first parameter is the image of the skin;
evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected;
matching lesion data recorded in a lesion database based on the skin lesion attribute to determine a pathological result of the object to be detected; and
outputting, by calling a second interface, the skin lesion attribute and the pathological result, wherein the second interface comprises a second parameter, and a value of the second parameter is the skin lesion attribute and the pathological result.

The above are only exemplary implementations of the present disclosure. It should be noted that for those of ordinary skill in the art, other improvements and modifications may further be made without departing from the principles of the present disclosure. These improvements and modifications should also fall within the scope of protection of the present disclosure.

1. A method of skin detection for an object, comprising:
acquiring an image of skin covering an outer surface of an object to be detected;
evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected; and
matching lesion data recorded in a lesion database based on the skin lesion attribute to determine a pathological result of the object to be detected.

2. The method of claim 1, wherein evaluating the image of the skin to determine the skin lesion region of the object to be detected comprises:
recognizing the image of the skin to obtain a region token sequence, wherein each region token in the region token sequence is used for representing a skin region of the object to be detected; and
extracting a sub-region token sequence from the region token sequence, wherein each region token in the sub-region token sequence is used for representing a sub-skin lesion region in the skin lesion region.

3. The method of claim 2, wherein extracting the sub-region token sequence from the region token sequence comprises:
converting the region token sequence into an image feature sequence of the image of the skin, wherein an image feature in the image feature sequence is used for representing the skin region corresponding to the region token in the region token sequence;
determining a sub-image feature sequence corresponding to the skin lesion region from the image feature sequence, wherein the skin lesion region comprises the skin region corresponding to the image feature in the sub-image feature sequence; and
determining the sub-region token sequence corresponding to the sub-image feature sequence.

4. The method of claim 2, wherein extracting the sub-region token sequence from the region token sequence comprises:
determining at least one region token having an importance level higher than a target threshold in the region token sequence as the sub-region token sequence, wherein the importance level is used for representing a level of importance of the corresponding region token to the pathological result.

5. The method of claim 4, wherein determining the at least one region token having the importance level higher than the target threshold in the region token sequence as the sub-region token sequence comprises:
selecting the at least one region token having the importance level higher than the target threshold from the region token sequence based on a lesion token selection module, wherein the lesion token selection module is at least configured to determine the importance level.

6. The method of claim 2, wherein generating the region token sequence for the image of the skin comprises:
partitioning the image of the skin into a plurality of the region tokens based on a vision transformer model to obtain the region token sequence, wherein the vision transformer model is obtained by training based on a self-attention mechanism.

7. The method of claim 2, wherein determining the skin lesion attribute of the object to be detected based on the skin lesion feature of the skin lesion region and the object type of the object to be detected comprises:
fusing the skin lesion features of the plurality of sub-skin lesion regions corresponding to the sub-region token sequence with the object type to obtain the skin lesion attribute.

8. The method of claim 1, further comprising:
calibrating the skin lesion attribute based on a target skin lesion attribute of the object to be detected, or calibrating the pathological result based on a target pathological result of the object to be detected, in order to match the skin lesion attribute with the pathological result.

9. A non-transitory computer-readable medium storing a set of instructions that is executable by at least one processor of an apparatus to cause the apparatus to perform a method of skin detection comprising:
acquiring an image of skin covering an outer surface of an object to be detected;
evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected; and
matching lesion data recorded in a lesion database based on the skin lesion attribute to determine a pathological result of the object to be detected.

10. The computer-readable storage medium of claim 9, wherein evaluating the image of the skin to determine the skin lesion region of the object to be detected comprises:
recognizing the image of the skin to obtain a region token sequence, wherein each region token in the region token sequence is used for representing a skin region of the object to be detected; and
extracting a sub-region token sequence from the region token sequence, wherein each region token in the sub-region token sequence is used for representing a sub-skin lesion region in the skin lesion region.

11. The computer-readable storage medium of claim 10, wherein extracting the sub-region token sequence from the region token sequence comprises:
converting the region token sequence into an image feature sequence of the image of the skin, wherein an image feature in the image feature sequence is used for representing the skin region corresponding to the region token in the region token sequence;
determining a sub-image feature sequence corresponding to the skin lesion region from the image feature sequence, wherein the skin lesion region comprises the skin region corresponding to the image feature in the sub-image feature sequence; and
determining the sub-region token sequence corresponding to the sub-image feature sequence.

12. The computer-readable storage medium of claim 10, wherein extracting the sub-region token sequence from the region token sequence comprises:
determining at least one region token having an importance level higher than a target threshold in the region token sequence as the sub-region token sequence, wherein the importance level is used for representing a level of importance of the corresponding region token to the pathological result.

13. The computer-readable storage medium of claim 12, wherein determining the at least one region token having the importance level higher than the target threshold in the region token sequence as the sub-region token sequence comprises:
selecting the at least one region token having the importance level higher than the target threshold from the region token sequence based on a lesion token selection module, wherein the lesion token selection module is at least configured to determine the importance level.

14. The computer-readable storage medium of claim 10, wherein generating the region token sequence for the image of the skin comprises:
partitioning the image of the skin into a plurality of the region tokens based on a vision transformer model to obtain the region token sequence, wherein the vision transformer model is obtained by training based on a self-attention mechanism.

15. The computer-readable storage medium of claim 10, wherein determining the skin lesion attribute of the object to be detected based on the skin lesion feature of the skin lesion region and the object type of the object to be detected comprises:
fusing the skin lesion features of the plurality of sub-skin lesion regions corresponding to the sub-region token sequence with the object type to obtain the skin lesion attribute.

16. The computer-readable storage medium of claim 9, wherein the set of instructions is executable by the at least one processor of the apparatus to cause the apparatus to further perform:
calibrating the skin lesion attribute based on a target skin lesion attribute of the object to be detected, or calibrating the pathological result based on a target pathological result of the object to be detected, in order to match the skin lesion attribute with the pathological result.

17. A system comprising:
a memory storing a set of instructions;
one or more processors configured to execute the set of instructions to cause the system to perform:
acquiring an image of skin covering an outer surface of an object to be detected;
evaluating the image of the skin to determine a skin lesion region of the object to be detected, wherein the skin lesion region is an image region with a skin lesion feature in the image of the skin;
determining a skin lesion attribute of the object to be detected based on the skin lesion feature of the skin lesion region and an object type of the object to be detected, wherein the skin lesion attribute is used for describing a skin lesion generated on the object to be detected; and
matching lesion data recorded in a lesion database based on the skin lesion attribute to determine a pathological result of the object to be detected.

18. The system of claim 17, wherein evaluating the image of the skin to determine the skin lesion region of the object to be detected comprises:
recognizing the image of the skin to obtain a region token sequence, wherein each region token in the region token sequence is used for representing a skin region of the object to be detected; and
extracting a sub-region token sequence from the region token sequence, wherein each region token in the sub-region token sequence is used for representing a sub-skin lesion region in the skin lesion region.

19. The system of claim 18, wherein extracting the sub-region token sequence from the region token sequence comprises:
converting the region token sequence into an image feature sequence of the image of the skin, wherein an image feature in the image feature sequence is used for representing the skin region corresponding to the region token in the region token sequence;
determining a sub-image feature sequence corresponding to the skin lesion region from the image feature sequence, wherein the skin lesion region comprises the skin region corresponding to the image feature in the sub-image feature sequence; and
determining the sub-region token sequence corresponding to the sub-image feature sequence.

20. The system of claim 18, wherein extracting the sub-region token sequence from the region token sequence comprises:
determining at least one region token having an importance level higher than a target threshold in the region token sequence as the sub-region token sequence, wherein the importance level is used for representing a level of importance of the corresponding region token to the pathological result.
